(19) United States
(12) Patent Application Publication
(10) Pub. No.: US 2008/0020088 A1
(43) Pub. Date: Jan. 24, 2008

(54) Novel Xylanases And Their Use

(76) Inventors: Jacques Georis, Liege (BE); Thierry Dauvrin, Couthuin (BE); Anne Hoyoux, Tilff (BE); Tony Collins, Angleur (BE); Georges Feller, Trooz (BE)

Correspondence Address: KNOBBE MARTENS OLSON & BEAR LLP, 2040 MAIN STREET, FOURTEENTH FLOOR, IRVINE, CA, 92614, US

(21) Appl. No.: 10/592,281

(22) PCT Filed: Mar. 11, 2005
(86) PCT No.: PCT/BE05/00035

§ 371 Date: Jul. 19, 2007

(30) Foreign Application Priority Data
Mar. 11, 2004 (EP) 04447062.3

(51) Int. Cl. C12N 9/24; A21D 13/00; A21D 2/08; A23G 3/00; A23J 1/00; A62D 3/02; C07H 21/00; D06M 16/00; C12C 11/00; C12N 1/00; C12N 1/15; C12N 1/19; C12N 1/21; C12N 15/00

(52) U.S. Cl. 426/11; 426/391; 426/653; 426/656; 426/658; 435/200; 435/243; 435/252.3; 435/254.11; 435/254.2; 435/262.5; 435/263; 435/267; 435/320.1; 536/23.2

(57) ABSTRACT

The present invention relates to novel enzymes with xylanolytic activity that belong to the glycoside hydrolase Family 8. The present invention in particular relates to enzymes isolated from bacterial psychrophilic strains that produce xylanases with an amino acid sequence as identified by any of SEQ ID NOs: 4, 6, 8, 10, 12, 14, 16, 35, 21, 23, 25, 27, 29, 31, 33, 37 or a variant thereof. Another aspect of the invention relates to the corresponding genes. These enzymes find many applications and are advantageously used for instance in feed and food applications such as baking. Compared to conventional xylanases, only small amounts of enzymes are needed to obtain a desired effect, such as an increase of the loaf volume and/or an increase in the width of cut on the surface of baked products.

### FIELD OF THE INVENTION

The present invention concerns novel enzymes with xylanolytic activity as well as their corresponding genes. In particular, the present invention concerns xylanases isolated from psychrophilic microorganisms and/or belonging to glycoside hydrolase Family 8. The enzymes of the invention find many applications and are highly suitable for use in bread improving compositions.

### BACKGROUND OF THE INVENTION

Xylans are heteropolysaccharides that form the major component of hemicellulose in plant biomass. The backbone of these polysaccharides consists of a chain of β-1,4-linked xylopyranoside residues. This backbone chain may be substituted to varying degrees with various side chain groups, including acetyl, arabinosyl and/or glucuronosyl substituents. Furthermore, phenolic compounds such as ferulic or hydroxycinnamic acids may also be involved, through ester binding, in the cross linking of the xylan chains or/and in the cross-linking of xylan and lignin chains.

Endoxylanases (also referred to as endo-β-1,4-xylanases, pentosanases or hemicellulases) specifically hydrolyse the backbone of xylan. In some cases, however, their activity may be sterically hindered by side groups. Different types of xylanases have been described and their specificity towards their substrate may vary from one to another, some being more active on insoluble arabinoxylans for instance. In addition, the length of the oligomers produced also depends on the type of xylanase considered.

Glycoside hydrolases have been classified into 93 families (http://afmb.cnrs-mrs.fr/CAZY/) based on sequence homologies and thus reflects structural and mechanistic features. Because the fold of proteins is better conserved than the sequence, some of the families can be grouped in ‘clans’ (Henrissat B. 1991, Biochem. J. Vol. 280, p. 309). Endo-beta-1,4-xylanases are generally classified in Families 10 (formerly Family F) and 11 (formerly Family G) and are found to frequently have an inverse relationship between their pI and molecular weight. The Family 10 xylanases (EXs10) are generally larger and more complex than the Family 11 xylanases (EXs11). Moreover, these families display significant differences in their structures and catalytic properties. EXs10 present an (α/β)₈ barrel fold, have about 40% α-helix structures and belong to clan GH-A (Dominguez et al. 1995, Nat. Struct. Biol. Vol. 2, p. 569) while EXs11 exhibit a β-jelly roll fold, have about 3-5% α-helix structures and belong to clan GH-C (Törrönen et al. 1994, EMBO J. Vol. 13 , p. 2493). EXs10 have a smaller substrate binding site and a lower substrate specificity. Furthermore, they frequently have endoglucanase activity and produce smaller oligosaccharides as compared to EXs11 (Biely et al. 1997, J. Biotechnol. Vol. 57, p. 151). Xylanases of both families as characterized to date retain the anomeric configuration of the glycosidic oxygen following hydrolysis in which two glutamates typically function as the catalytic residues (Jeffries, 1997, Curr. Opin. Biotechnol. Vol. 7, p. 337).

Xylanases are used in various industrial sectors including the food, feed and some technical industries.

In the food industry, xylanases are used in fruit, vegetable and plant processing, in wine making and brewing, in baking, milling, pastry and confectionery and in coffee processing. Their functions in these industries are very diverse. For example, in fruit and plant processing they improve the maceration process, juice clarification, the extraction yield and filtration efficiency, hence improving the process performance and product quality. Xylanases also reduce the wart viscosity in beer making, improve grape skin maceration in wine making and reduce haze in the final products. In baking, xylanases improve elasticity and strength of doughs, thereby allowing easier handling, larger loaf volume and improved bread texture. In coffee processing, xylanases reduce the viscosity of coffee extracts and improve the drying/lyophilization processes.

Xylanases are further used in feed for monogastric animals (swine and poultry) and ruminants. They decrease among others the content of non-starch polysaccharides, thereby reducing the intestinal viscosity and improving the utilization of proteins and starch present in the feed. Xylanases improve animal performance and increase the digestibility and nutritive value of poorly degradable feeds such as barley and wheat.

In the starch industry, the use of xylanases improves the gluten-starch separation process.

In the pulp and paper industry, xylanases are used to facilitate the pulping process and to reduce the use of mechanical pulping methods, hence reducing energy consumption. Xylanases also improve the fibrillation and drainage properties of pulp, hence improving the process efficiency and the paper strength. They also facilitate the de-inking processes and reduce the use of alkali in these processes.

Xylanases are further used in the enzymatic retting of textiles (flax, jute, ramie, hemp, . . . ) and therefore reduce or replace chemical retting methods. They are also used in bioremediation for the treatment of agricultural and food industry wastes. Some bioconversion processes such as the production of fermentable products, renewable fuel (bioethanol) or fine chemicals for instance are also performed with the aid of xylanases.

Further applications for xylanases may still arrive in the future.

The use of xylanases (also referred to as endoxylanases, endo-β-1, 4-xylanases, pentosanases or hemicellulases) in baking has been well known for a number of years. These dough-conditioning enzymes can improve the dough machinability and stability as well as the oven-spring and the crumb structure. Other effects of the enzymes are a larger loaf volume and a softer crumb.

The mechanism of action of xylanases in bread preparation is still not clearly elucidated. Wheat flour contains about 3 to 4% pentosans. These pentosans can absorb large amounts of water (up to 30%) and this water absorption contributes to the properties of the dough as well as to the quality of the final product. Partial hydrolysis of pentosans by pentosanases into water soluble short chain oligosaccharides increases the water binding capacity. In addition, the pentosans strongly interact with the gluten fraction of the flour to form a network. Pentosanases may help to relax this strong and rigid network, thereby allowing better dough expansion by the carbon dioxide formed by the yeast.

Many types of hemicellulase preparations have been used for the applications mentioned above, and are commercially available. They are produced by microbial fermentation using various microorganisms as enzyme sources. Some of these enzymes are also produced by genetically modified microorganisms. All documented commercial uses of xylanases relate to xylanases belonging to either glycoside hydrolase Family 10 or Family 11, as defined previously.

Examples of commercial xylanases are the xylanases from Bacillus sp., Trichoderma sp., Humicola sp. and Aspergillus sp.

A number of reviews describing the actual state of art in the field of xylanases have been recently published (see for example Shallom D & Shoham Y. 2003, Curr. Opin. Microbiol. Vol. 6, p. 219- Subramaniyan S & Prema P. 2002, Crit Rev Biotechnol. Vol 22, p. 33-Beg Q K, Kapoor M, Mahajan L & Hoondal G S. 2001, Appl Microbiol Biotechnol. Vol. 56, p. 326).

Xylanases belonging to families other than glycoside hydrolase Families 10 and 11 have been recently described (see e.g. http://afmb.cnrs-mrs.fr/CAZY/ with the EC code for xylanase 3.1.2.8. for an overview). Among these, one example is the xylanase from Pseudoalteromonas haloplanktis TAH3a belonging to glycoside hydrolase Family 8 which has been characterized and for which the corresponding gene has been cloned (Collins T. et al. 2002, J. Biol. Chem. Vol. 277, p. 35133; Collins T. et al. 2003, J. Mol. Biol. Vol 328, P. 419; Van Petegem F. et al. 2003, J. Biol. Chem. Vol 278, p. 7531). This enzyme is a typical psychrophilic enzyme and presents a high catalytic activity at low temperatures. It is not homologous to family 10 or 11 xylanases, but has 20 to 30% identity with glycoside hydrolase Family 8 members (formerly Family D), a family that comprises mainly endoglucanases, but also lichenases and chitosanases. Furthermore, a FingerPRINTScan against PRINTS using the InterPro Scan search program (Zdobnov and Apweiler, 2001, Bioinformatics Vol. 17, p. 847) indicated that the isolated sequence contained the glycosyl hydrolase Family 8 fingerprint. In addition, the isolated sequence contains Family 8 residues that are strictly conserved in the 53 Family 8 enzymes analyzed thus far.

In contrast to most EXs10 and EXs11, this Family 8 xylanase (EXs8) has both a high pI and a high molecular weight. Structural and catalytic properties are different from those of both EXs10 and EXs11. The EXs8 xylanase presents a distorted (α/α)₆ barrel fold with 13 α-helices and 13 β-strands and belongs to clan GH-M (Van Petegem et al. 2003, J. Biol. Chem. Vol. 278 (9), p7531-9) . This enzyme has no endoglucanase, chitosanase or licheninase activity and appears to be functionally similar to EXs11, being more active on long chain xylo-oligosaccharides.

In contrast to other characterized EXs10 and EXs11 that retain the anomeric configuration, Family 8 glycoside hydrolases (Fierobe et al., 1993. Eur. J. Biochem. Vol. 217, p557; http://afmb.cnrs-mrs.fr/CAZY/) tend to hydrolyse the substrate with inversion of the anomeric configuration of the glycosidic oxygen. This has been shown for the psychrophilic xylanase from Pseudoalteromonas haloplanktis (Collins, T. et al. 2002. J. Biol. Chem. Vol. 277, p. 35133). In addition, the catalytic residues involved in hydrolysis are believed to be a glutamate and an aspartate.

A variety of methods exist for evaluating the xylanase activity in an enzyme preparation. Examples of such methods for xylanase activity determination include: the measurement of the release of reducing sugars from xylan (Miller G. L. 1959, Anal. Chem. Vol. 31, p. 426), or the measurement of the release of coloured compounds from modified substrates (for examples AzoWAX or Xylazyme AX from Megazyme). However, no direct correlation has been shown between the xylanolytic activity found in various enzyme preparations and the effect in a particular application. For example in baking, dose-related results can be observed to a certain extent for a single enzyme, but the same dosage of two xylanases of different origins does not give the same result in the dough or in the bread. Several reasons could explain this: differences in substrate specificity, differences in temperature and pH optimum, differences in catalytic efficiency, . . .

It is of great interest to develop novel enzyme preparations, such as, but not restricted to ingredients for bread improver compositions or agents with new or improved properties. One of these properties could be that the xylanase content is as low as possible (in terms of weight of enzymes needed to obtain a particular result).

### AIMS OF THE INVENTION

The present invention aims to provide novel enzymes and enzyme preparations with xylanolytic activity.

The present invention further aims to provide amino acid and nucleotide sequences encoding such enzymes and their variants.

Another aim of the invention is to provide enzymes and enzyme preparations for use in agrofood, feed and many other technical processes.

Yet another aim of the present invention is to provide enzymes and enzyme preparations with xylanolytic activity of which the amount needed to observe a specific effect in a particular application is far lower than for any conventional commercial enzymes.

Still another aim of the present invention is to provide enzymes and enzyme preparations with xylanolytic activity, which are remarkably more stable than enzymes known in the art, and which preferably are more effective at the same time (reflected by a significantly lower amount needed to obtain a given effect).

### SUMMARY OF THE INVENTION

The present invention is directed to novel xylanolytic enzymes that preferably belong to glycoside hydrolase Family 8 and preferably are psychrophilic enzymes, most preferably psychrophilic enzymes that hydrolyse with inversion of the anomeric configuration, with the proviso that said xylanolytic enzyme is not encoded by SEQ ID NO: 1.

Advantageously, the enzymes of the present invention increase the loaf volume of a baked product by at least 10% when applied in a concentration of 1500 to 6000 xylanase units/100 kg flour and/or increase the width of cut on the surface of a baked product when applied in a concentration of 1500 to 6000 xylanase units/100 kg flour. The enzymes of the present invention may be advantageously used in frozen doughs and/or in applications that involve low temperature proofing.

Preferably, the enzymes comprise an amino acid sequence, or consist of an amino acid sequence, corresponding to any of SEQ ID NOs: 4, 6, 8, 10, 12, 14, 16 or 35. These sequences contain 426 amino acids (aa), of which the first 21 constitute a signal peptide. A preferred sequence is SEQ ID NO: 4, representing the xylanase isolated from TAH 2a.

Preferably, said enzymes—identified by one of the above SEQ ID NOs—belong to glycoside hydrolase Family 8 and are preferably psychrophilic enzymes.

Another aspect of the invention concerns the mature polypeptides of any of the above, corresponding to aa 22-426 of any of the above sequences. Prefered amino acid sequences according to the invention are SEQ ID NOs: 21, 23, 25, 27, 29, 31, 33 and 37 and SEQ ID NOs: 20, 22, 24, 26, 28, 30, 32 and 36, the corresponding nucleotide sequences encoding such mature polypeptides.

The enzymes of the invention may also have an amino acid sequence that differs in less than 10, 9, 8, 7, 6, 5, 4, 3, 2 or 1 aa from any of the above (mature protein or propeptide) , or have an amino acid sequence that has more than 50%, 70%, more preferably more than 80%, 85%, 90%, most preferably more than 95%, 97%, 98% or even more than 99% sequence identity with any of SEQ ID NOs: 2, 4, 6, 8, 10, 12, 14, 16, 35, 21, 23, 25, 27, 29, 31, 33 or 37.

The enzymes of the invention preferably are isolated from any of the following bacterial strains: TAH 2a, TAH 4a, TAH 7a, Sp.10.1, Sp.10.2, Sp.11.2, Sp.23.2 or SP.23.2bis. These strains concern another aspect of the invention.

Another aspect of the invention concerns nucleotide sequences that encode any of the above xylanolytic enzymes.

A preferred such nucleotide sequence is one that comprises SEQ ID NO: 19 and codes for a TAH 2a, TAH 4a, TAH 7a, Sp.10.1, Sp.10.2 or Sp.11.2 xylanase as identified by any of SEQ ID NOs: 4, 6, 8, 10, 12, 14, 21, 23, 25, 27, 29 or 31.

Other preferred nucleotide sequences are those coding for one of the above-identified preproteins or mature polypeptides. Examples of such nucleotide sequences include SEQ ID NOs: 3, 5, 7, 9, 11, 13, 15, 34, 20, 22, 24, 26, 28, 30, 32 and 36.

A further aspect of the present invention concerns recombinant nucleotide sequences that comprise one or more of the above nucleotide sequences, operably linked to one or more adjacent regulatory sequence(s). Another aspect of the invention concerns vectors harboring the latter and host cells transformed therewith.

A preferred embodiment of the invention is directed to a plasmid incorporated in Escherichia coli having a deposit number selected from the group consisting of LMBP 4860, LMBP 4861, LMBP 4862, LMBP 4863, LMBP 4864, LMBP 4865 and LMBP 4866. LMBP 4862 is preferred.

The enzymes of the invention may be extra-cellularly expressed or may be intracellularly expressed. It is possible to use directly cells or cell cultures that express the enzymes according to the invention and/or to use the supernatant only of cell cultures in which the enzymes are secreted. Alternatively, cell extracts and/or cell-free extracts may be used as source of the enzymes of the invention. Finally, the enzymes may be used after being purified or partly purified (after at least one purification step), id est in more or less pure form.

The xylanases of the invention are highly suitable for use in applications that involve the degradation of plant cell wall components. They are for instance advantageously used in processes for decomposing plants and fruits, preferably fruit, legume juice, beer, paper, starch, gluten or vegetable oil preparation processes. Other applications include their use in fruit, vegetable and plant processing, wine making or brewing, coffee processing, the processing of paper or textile, in bioconversion processes, in processes for decomposing wastes, preferably for decomposing agricultural wastes or wastes from paper mills, in starch-gluten separation processes, in feed preparations, in baking, milling, pastry and confectionery processes.

The enzymes of the invention are particularly suitable for use in food applications.

For some applications it is advantageous to fix the above described cells, components of the above cell extracts and/or one or more purified enzymes with xylanolytic activity according to the invention to a solid support.

The enzymes of the invention may be used in a bread improver or bread improving composition. Apart from one or more xylanolytic enzymes according to the invention, said improver or improving composition may further contain at least one other bread-improving agent selected from the list consisting of other enzymes, emulsifiers, oxidants, milk powder, fats, sugars, amino acids, salts and proteins (gluten, cellulose binding sites) or a mixture thereof. Said other enzyme preferably is selected from the list consisting of alpha-amylases, beta-amylases, maltogenic amylases, other xylanases, proteases, glucose oxidase, oxido-reductases, glucanases, cellulases, transglutaminases, isomerases, lipases, phospholipases, pectinases or a mixture thereof. Said alpha-amylase preferably is an alpha-amylase obtained from Aspergillus oryzae.

Preferably the above-described bread improving composition is added during the mixing of the dough.

The enzymes of the invention may be combined with any other enzyme or ingredient and may be used in the form of for instance a dry powder or a granulate, in particular a non-dusting granulate. Alternatively, they may be used in the form of a liquid, preferably with one or more stabilizer(s) such as polyols, sugars, organic acids or sugar alcohols.

A last aspect of the invention concerns compositions comprising at least one, preferably at least two or three of the enzymes of the invention, possibly combined with a Pseudoalteromonas haloplanktis TAH 3a xylanase, encoded for instance by SEQ ID NO:1.

### BRIEF DESCRIPTION OF THE DRAWINGS

FIGS. 1a and b represent, respectively, the nucleotide (a) and the amino acid (b) sequences of the Pseudoalteromonas haloplanktis TAH 3a xylanase.

FIG. 2 represents the thermodependendency of the xylanolytic activity of TAH 2a (circles, continuous line), TAH 4a (squares, dashed line) and TAH 7a (triangles, short dashed lines.).

FIGS. 3a and b represent, respectively, the nucleotide (a) and the amino acid (b) sequences of the strain TAH 2a xylanase.

FIGS. 4a and b represent, respectively, the nucleotide (a) and the amino acid (b) sequences of the strain TAH 4a xylanase.

FIGS. 5a and b represent, respectively, the nucleotide (a) and the amino acid (b) sequences of the strain TAH 7a xylanase.

FIGS. 6a and b represent, respectively, the nucleotide (a) and the amino acid (b) sequences of the strain Sp.10.1 xylanase.

FIGS. 7a and b represent, respectively, the nucleotide (a) and the amino acid (b) sequences of the strain Sp.10.2 xylanase.

FIGS. 8a and b represent, respectively, the nucleotide (a) and the amino acid (b) sequences of the strain Sp 11.2 xylanase.

FIGS. 9a and b represent, respectively, the nucleotide (a) and the amino acid (b) sequences of the strain Sp.23.2 xylanase.

FIG. 10 shows an alignment of the nucleotide sequences of the xylanases from strains TAH 3a, TAH 2a, TAH 4a, TAH 7a, Sp.10.1., Sp.10.2., Sp.11.2., Sp.23.2 & Sp.23.2bis. The symbol * under the alignment shows identical nucleotides in all sequences listed.

FIG. 11 shows an alignment of the amino acid sequences of the xylanases from strains TAH 3a (xyl3), TAH 2a, TAH 4a, TAH 7a, Sp.10.1., Sp.10.2., Sp.11.2., Sp.23.2 & Sp.23.2bis. The symbol * under the alignment shows identical amino acids in all sequences listed.

FIG. 12 shows the results of an SDS polyacrylamide gel electrophoresis with Sp.11.2, Sp.23.2bis, TAH 2a and TAH 7a purified xylanases.

FIG. 13 shows the relationship between the relative amounts of protein (xylanase) and the performance in baking of different xylanases according to the invention.

FIG. 14 shows a preferred nucleotide sequence: SEQ ID NO: 19.

FIG. 15 shows volume-response curve for the xylanases of the invention in a baking test.

FIGS. 16a and b represent, respectively, the nucleotide (a) and the amino acid (b) sequences of the strain Sp.23.2bis xylanase

The invention will be described in further details in the following examples and embodiments by reference to the enclosed drawings. Particular embodiments and examples are not in any way intended to limit the scope of the invention as claimed.

### DETAILED DESCRIPTION OF THE INVENTION

A. Isolation of the Enzyme Producing Microorganisms according to the Invention

A first aspect of the invention is related to the isolation of uncharacterized microorganisms able to produce xylanases with new properties.

Advantageously, these microorganisms are psychrophilic microorganisms isolated from environmental samples collected in the Antarctic and Arctic environments. Methods are described in the literature that relate to the isolation of psychrophilic microorganisms from such samples (Collins T. et al., 2002, J. Biol. Chem. Vol. 277, p. 35133). Typically these methods consist of spread-plating appropriate dilutions of the samples onto solid media of various compositions. Incubations are generally performed at low temperatures (about 4 to about 20° C.), to allow the growth of the psychrophilic microorganisms.

According to a preferred embodiment of the present invention, the solid medium contains a substrate for xylanase such as a hemicellulose-containing material or xylan.

According to a more preferred embodiment of the invention, the solid medium contains a derivatized substrate for the xylanase such as for example RBB-xylan (Remazol Brilliant Blue xylan, Sigma). This substrate allows a better, generally more sensitive, screening of the positive xylanase-producing microorganisms.

It is obvious that techniques other than those described here, exist for the screening of xylanase-positive microorganisms. One such technique involves systematic liquid culturing of microorganisms using e.g. media that contain an inducer for the xylanase such as, but not restricted to, xylan, wheat bran, etc., followed by enzymatic analysis of the culture supernatant and/or of a cell extract.

According to another embodiment of the present invention, the isolated xylanase-positive microorganisms may or may not be identified at the genus or the species level. Techniques such as fatty acid analysis or 16S rDNA sequence analysis are examples of well known identification techniques.

The present invention is more in particular related to isolated strains TAH 2a, TAH 4a, TAH 7a, Sp10.1, Sp10.2, Sp11.2, Sp23.2 and Sp23.2bis.

TAH2a, TAH4a and TAH7a are bacterial strains, more specifically bacterial strains from the genus Pseudoal teromonas.

B. Isolation of Xylanase Encoding Genes

The present invention relates to the isolation of xylanase encoding genes from the above mentioned strains.

It is well know to persons skilled in the art that several techniques may be used to isolate a particular gene from microorganisms. Examples of such techniques include the construction of a gene library from the genomic DNA of the strain in a suitable vector, followed by screening of this library by direct expression of the gene of interest, or by hybridization with a presumed closely related gene or with oligonucleotides designed from reverse transcription of the partial or complete amino acid sequence of the said enzyme.

Other techniques include the amplification of the gene by molecular techniques, for instance PCR techniques, using degenerate or non-degenerate oligonucleotide primers that are designed from a presumed closely related gene.

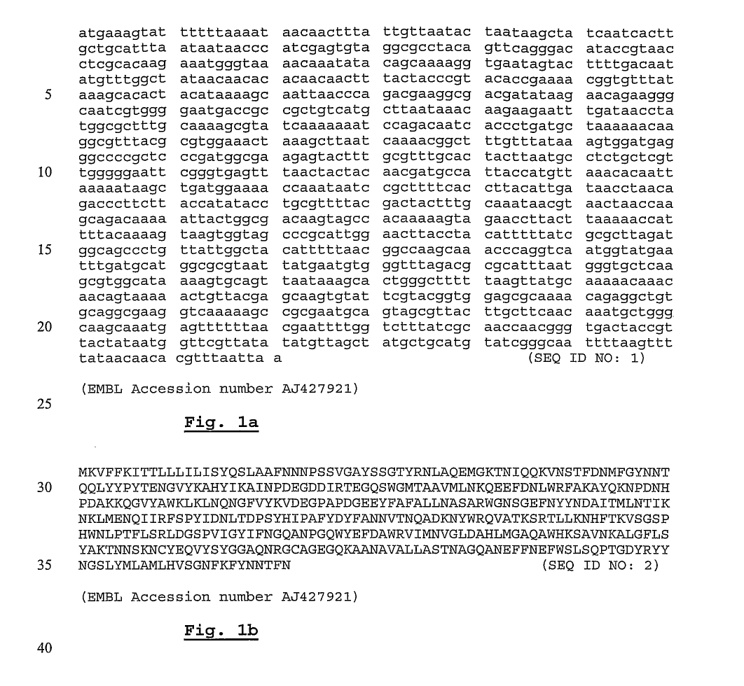

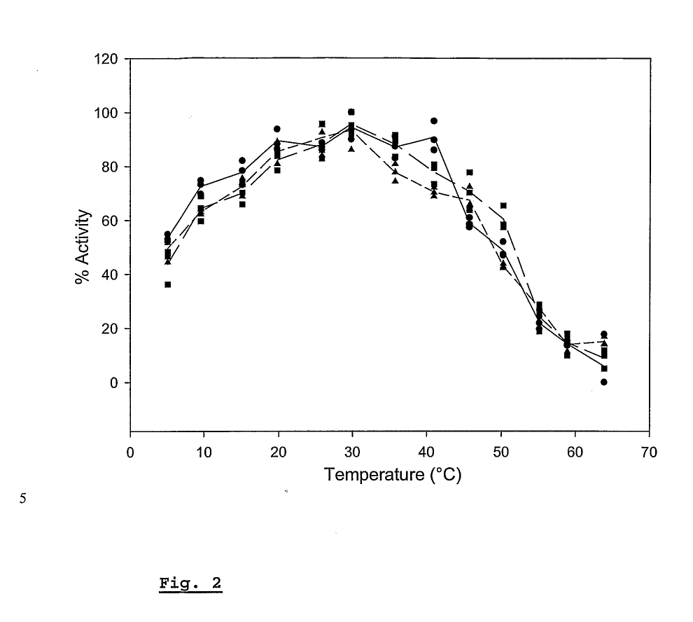

According to a preferred embodiment of the present invention, the starting sequence used to design such oligonucleotide primers is the sequence of the xylanase gene isolated from Pseudoalteromonas haloplanktis TAH 3a (EMBL Accession number AJ427921, sequence enclosed by reference thereto, see also FIG. 1a or SEQ ID NO 1).

An aspect of the invention is related to isolated (and purified) nucleotide sequences SEQ ID NOs: 3, 5, 7, 9, 11, 13, 15, 34, 20, 22, 24, 26, 28, 30, 32, 36 or variants thereof that encode an enzyme according to the invention.

“Variant”, in the present context, is defined as an isolated nucleotide sequence wherein one, or several, nucleotides have been replaced by other nucleotide(s) or wherein one, or several, nucleotides have been added and/or deleted while the encoding enzyme still presents xylanolytic activity.

According to a preferred embodiment of the present invention, the nucleotide sequence of the variant presents more than 50%, 60%, 70%, 80%, 85%, 90%, 95%, preferably more than 97%, 98% or even more than 99% sequence identity to the isolated sequences. According to a more preferred embodiment of the present invention, the nucleotide sequence of the variant presents less than 20, 19, 18, 17, 16, 15, 14, 13, 12, 11, 10, 9, 8, 7, 6, 5, 4, 3, 2 or 1 modified nucleotide(s) compared to SEQ ID NOs: 3, 5, 7, 9, 11, 13, 15, 34, 20, 22, 24, 26, 28, 30, 32 or 36.

C. Characterization of the Xylanases of the Invention

Another aspect of the present invention is related to enzymes with xylanolytic activity encoded by any of the nucleotide sequences described above.

According to a preferred embodiment of the present invention, the amino acid sequence of such an enzyme is selected from the group consisting of SEQ ID NOs: 4, 6, 8, 10, 12, 14, 16, 35, 21, 23, 25, 27, 29, 31, 33, 37 or a variant thereof.

In the present context, “variant” is defined as an isolated amino acid sequence wherein one, or several, amino acids have been substituted by one, or several, others, or as an isolated amino acid sequence wherein one, or several, amino acids have been added and/or deleted while preserving all or most of the xylanase activity. Variant enzymes preferably retain at least 80%, more preferably at least 90% and most preferably at least 95% or even 99% of the xylanolytic activity of any of the enzymes characterized by SEQ ID NO: 4, 6, 8, 10, 12, 14, 16, 35, 21, 23, 25, 27, 29, 31, 33 or 37.

According to a preferred embodiment of the present invention, the amino acid sequence of the variant presents more than 50%, 70%, more preferably more than 80%, 85%, 90%, most preferably more than 95%, 97%, 98% or even more than 99% sequence identity with any of SEQ ID NOs: 4, 6, 8, 10, 12, 14, 16, 35, 21, 23, 25, 27, 29, 31, 33 or 37. According to a more preferred embodiment of the present invention, the amino acid sequence of the variant presents less than 4, 3, 2 or 1 modified amino acid(s) compared to any of SEQ ID NOs: 4, 6, 8, 10, 12 14, 16, 35, 21, 23, 25, 27, 29, 31, 33 or 37.

It is well known that most secreted enzymes, such as the enzymes of the present invention, possess a small amino acid sequence at their N terminus that drives the enzyme out of the cell. It is therefore an objective of the present invention to also consider the mature portions of the enzymes as an integral part of the present invention (see e.g. SEQ ID NOs: 21, 23, 25, 27, 29, 31, 33 or 37). However, variants containing modifications in the signal sequence could advantageously be used, as these could eventually be better expressed and/or be better secreted in a recombinant host.

The mature protein starts at amino aid position No. 22 for the enzymes identified by any of SEQ ID NOs: 2, 4, 6, 8, 10, 12, 14, 16 or 35.

Another means of characterizing proteins and/or enzymes is to study their identity or their similarity with already known proteins. A first approach could be to use the Blast program that compares a defined sequence to all available published sequences. This program can be used on-line at the following URL (http://www.ncbi.nlm.nih.gov/blast/). To refine and precise the percentage of identity/similarity between two or more proteins the programs Clustal (http://www.ebi.ac.uk/clustalw/) or Align (http://www2.igh.cnrs.fr/bin/align-guess.cgi) may advantageously be used.

A further means of characterizing new proteins and/or enzymes is to analyze their three-dimensional structures. In some cases, this three-dimensional structure is not known. However, by comparing with other available structures of related proteins, this three-dimensional structure could be deduced by a person skilled in the art by using for instance specialized software. Furthermore, for very closely related proteins, e.g. proteins with more than 90% identity/similarity, the changes in properties caused by a particular modification in the amino acid sequence (e.g. amino acid substitution) could be predicted. Such changes could be e.g. substrate binding, protein stabilisation, . . .

Another aspect of the present invention concerns the characterization of the xylanases of the invention by their biochemical properties such as their molecular weight, their temperature optimum or pH optimum. Indeed the enzymes described in the present invention, by means of the examples below, have a relatively low temperature optimum activity. Furthermore, they retain a high catalytic efficiency at low temperature and are therefore considered as psychrophilic xylanases. One way of defining a psychrophilic enzyme is to consider that it is an enzyme that retains at least 40% of its maximal activity at 5° C. Other properties may also be used to define psychrophilic enzymes (see e.g. Georlette D. et al., 2004, FEMS Microbiol. Rev. Vol 28, p. 25 ; D'Amico S. 2002, Philos Trans R Soc Lond B Biol Sci. Vol. 357, p.917).

However, it can not be excluded that there exist enzymes with the sequence characteristics described above that have different biochemical properties.

D. Expression and/or Purification of the Enzymes

Another aspect of the present invention is related to a recombinant nucleotide sequence comprised of one or more adjacent regulatory sequence(s) operably linked to one or more of the nucleotide sequence(s) of the invention as described above.

Said adjacent regulatory sequences may originate from homologous and/or from heterologous microorganisms.

These adjacent regulatory sequences are specific sequences such as but not restricted to promoters, regulators, secretion signals and/or terminators.

Another aspect of the invention is related to a vector containing one or more nucleotide sequences of the invention, possibly operably linked to one or more adjacent regulatory sequence(s) which originate from homologous or/and from heterologous microorganisms.

In the present context, “vector” is defined as any biochemical construct which may be used for the introduction of a nucleotide sequence (by transduction, transfection, transformation, infection, conjugation, etc.) into a cell. Advantageously, the vector described in the invention is selected from the group consisting of plasmids, viruses, phagemids, chromosomes, transposons, liposomes, cationic vesicles, and/or a mixture thereof. Said vector may already contain one or more of the above-described adjacent regulatory sequence(s) (allowing its expression and its transcription into a corresponding peptide by said microorganism). Preferably, said vector is a plasmid incorporated in E. coli and having a deposit number selected from the group consisting of numbers LMBP 4860, LMBP 4861, LMBP 4862, LMBP 4863, LMBP 4864, LMBP 4865 and LMBP 4866.

The present invention is also related to a host cell, preferably a recombinant host cell, “transformed” with one or more of the nucleotide sequences and/or vectors according to the invention that have been described above.

In the present context, “host cell “transformed by one or more of the nucleotide sequences and/or vectors according to the invention” is defined as a cell having incorporated said nucleotide sequence(s) and/or said vector. The transformed host cell may be a cell in which said vector and/or said nucleotide sequence(s) are introduced by means of genetic transformation, preferably by means of homologous recombination, or by another well known method to generate/create a recombinant organism.

A “host cell” may be a cell that, prior to transformation, did not naturally (originally) contain said nucleotide sequence(s) and/or said vector. Alternatively, the “host cell” may also be the original cell containing already one of the nucleotide sequences of the present invention, and genetically modified (recombinant host cell) to overexpress, or express more efficiently, said enzyme (better pH or temperature profile, higher extracellular expression, . . . ).

Preferably, said host cell is capable of overexpressing (higher expression than the expression observed in the original or wild-type microorganism) said nucleotide sequence(s) and/or said vector, advantageously allowing a high production of an amino acid sequence encoded by said nucleotide sequence and/or said vector. The isolated and purified nucleotide sequence(s) described in the present invention may be integrated into the genome of the selected host cell and/or may be present on an episomal vector in said host cell.

Advantageously, the recombinant host cell of the invention is selected from the microbial world, preferably from bacteria or fungi, including yeast.

More preferably the recombinant host cell belongs to the genus Bacillus.

Preferably, said recombinant host cell is modified to obtain an expression of the xylanase enzyme at high levels. This may be obtained by the use of adjacent regulatory sequences capable of directing the overexpression of one or more of the nucleotide sequences of the invention in the recombinant host cell, or by increasing the number of nucleotide copies of the sequences according to the invention.

Above, some of the conditions (culture media, temperature and pH conditions, etc.) have been described that preferably are applied for culturing of the host cell selected for the expression of the xylanase of the invention. For this purpose, the original production species and/or a suitable host cell, transformed with a DNA construct designed to express the said enzyme, are present in or on a suitable growth medium and/or expression medium.

According to the present invention, said protein with xylanolytic activity may be obtained by first culturing the strain in/on a medium suitable for expressing the xylanase, followed by one or several purification steps, such as, but not limited to, centrifugation, cell disruption, microfiltration, ultrafiltration, precipitation, liquid chromatography, freeze-drying, etc. All these techniques are described in the scientific literature and are well known to persons skilled in the art.

Accordingly, the enzyme with xylanolytic activity which is produced according to the present invention, can be used directly for one of the purposes of the present invention and/or, alternatively, may undergo one or more purification and/or (further) culturing steps as described above. In a particular embodiment of the invention, the enzyme(s) of the invention may be used in pure form.

Preparation methods for the enzyme of the present invention include, among others, the preparation and/or purification from cultures of microorganisms, recombinant or not, in shake flasks or in fermentors, and the preparation or purification from immobilized cultures as well as the extraction and/or purification from living cells (plants, . . . )

E. Application of the Enzyme

The xylanase of the invention may be used in different types of applications.

The enzymes with xylanolytic activity described in the present invention, purified or not purified, are particularly suited as bread improving agents or formulas. Bread improving agents or compositions are products that are able to improve and/or increase texture, flavour, anti-staling effects, softness, crumb softness upon storage, freshness, dough machinability and/or volume of a dough and/or a final baked product. Preferably, said enzymes with xylanolytic activity improve the dough handling and/or increase the specific volume of the final baked product.

The term “baked product” includes any product prepared from a dough and obtained after baking of the dough, and includes in particular yeast raised baked products. Dough is obtained from any type of flour or meal (e.g. based on wheat, rye, barley, oat, or maize). Preferably, dough is prepared with wheat and/or with mixes including wheat.

It is one of the aspects of the present invention to show that the enzymes with xylanolytic activity described in the present invention could advantageously be used in a bread improver formula or bread improving composition.

These enzymes with xylanolytic activity advantageously are selected from glycoside hydrolase Family 8 enzymes.

These enzymes with xylanolytic activity advantageously are psychrophilic enzymes.

It is shown in sequel that said enzymes increase the loaf volume of a baked product.

It is further shown that the amount of said enzymes, needed to obtain a particular result in baking, is much lower than the amount generally used/needed when working with commercially available enzymes. Such amount is further referred to as the amount sufficient to obtain the desired result and/or as the effective amount.

In a further aspect, the present invention relates to the additive effect of said enzymes with xylanolytic activity with other enzymes, in particular with an alpha-amylase, preferably an alpha-amylase from Aspergillus oryzae. Said enzymes with xylanolytic activity may be used in combination with other bread improving agents, such as, but not limited to enzymes, emulsifiers, oxidants, milk powder, fats, sugars, amino acids, salts, proteins (gluten, cellulose binding sites), such improving agents being well known to persons skilled in the art. Examples of such enzymes include, but are not restricted to, alpha-amylases, beta-amylases, maltogenic amylases, xylanases, proteases, glucose oxidases, oxido-reductases, glucanases, cellulases, transglutaminases, isomerases, lipases, phospholipases, pectinases, etc.

According to the present invention, the enzymes with xylanolytic activity, purified or not, show hydrolytic activities in the presence of cell wall components. Particularly, said enzymes degrade the wheat cell wall components. Said enzymes may thus be advantageously used in the separation of components of plant cell materials, such as cereal components. Particularly, said enzymes may be used to improve the separation of wheat into gluten and starch by the so-called batter process.

According to the present invention, the enzymes with xylanolytic activity, purified or not, may be used in food processing. They may be used in fruit, vegetable and plant processing. They may be used in wine making and brewing as well as in coffee processing.

Another application of said enzymes resides in feed, where they may be used to improve the growth rate or the feed conversion ratio of animals such as poultry, swine or ruminants.

Yet other applications are the use of the xylanases of the invention in the pulp and paper industry (bio-mechanical pulping, bio-modification of fibers, bio-de-inking, . . . ), in textile processing, in bioremediation, in bioconversion processes,.

The enzymes with xylanolytic activity according to the present invention may be used under several forms. Cells expressing the enzyme, such as yeast, fungi, Archeal bacteria or bacteria, may be used directly in the process. Said enzymes may further be used as a cell extract, a cell-free extract (i.e. portions of the host cell that have been submitted to one or more disruption, centrifugation, extraction and/or other purification steps) or as a purified protein. Any of the above-described forms may be used in combination with one or more enzyme(s) under any of the above-described forms. These whole cells, cell extracts, cell-free extracts and/or purified enzymes may be immobilized by any conventional means on a solid support to allow protection of the enzyme, continuous hydrolysis of the substrate and/or recycling of the enzyme preparation. Said cells, cell extracts (including crude and partially purified extracts), cell-free extracts and/or enzymes may be mixed with different ingredients, and be used e.g. in the form of a dry powder or a granulate, in particular a non-dusting granulate, in the form of a liquid, for example with stabilisers such as polyols, sugars, organic acids or sugar alcohols according to established methods.

### EXAMPLES

### Example 1

### Isolation and Characterization of Xylanase Producing Microorganisms

The xylanase producing microorganisms were isolated from either wood samples collected in the vicinity of the abandoned French Antarctic station in Port Martin, Terre Adelie, Antarctica (66°40′S; 140°01′E) or from sea water samples collected in the vicinity of Ny-Alesund, Spitzberg, Svalbard, Norway. Screening for xylanase activity was carried out on marine agar (5 g/l tryptone (Difco), 1 g/l yeast extract (Difco), 33 g/l marine salts (Wiegandt), 18 g/l agar (Difco)) supplemented with 0.15% Remazol Brilliant Blue-xylan at 4° C. A clear halo appeared around the xylanase producing microorganisms, allowing the isolation of 7 strains. Those isolated from Antarctica were designated TAH 2a, TAH 4a, TAH 7a, and those isolated from Spitzberg were termed Sp.10.1, Sp.10.2, Sp.11.2, Sp.23.2 and Sp.23.2bis.

The cellular fatty acid composition of strains TAH 2a, TAH 4a and TAH 7a has been determined. These three strains belong most probably to the Pseudoalteromonas sp.

### Example 2

### Production of Wild-Type Xylanases Culture Conditions

The isolated xylanase producing bacterial strains TAH 2a, TAH 4a, TAH 7a, Sp.10.1, Sp.10.2, Sp.11.2, Sp.23.2 and Sp.23.2bis were cultivated in modified marine broth (5g/liter tryptone (Difco), lg/liter yeast extract (Difco), 20g/liter marine salts (Wiegandt)) supplemented with 15g/liter birchwood xylan (Sigma) (in the case of TAH2a, TAH7a, Sp.10.1, Sp10.2, Sp.11.2, Sp.23.2 and Sp.23.2bis) or 15 g/liter wheat hemicellulose (Beldem) (in the case of TAH 4a) for 72 hours at 4° C.

Preparation of the Enzymes

After centrifugation of the above cultures (see culture conditions above) for 1 h at 18,000× g and 4° C., the supernatant was concentrated by precipitation with 80% ammonium sulphate and resuspended in 20 mM MOPS (3-(N-Morpholino)propanesulfonic acid) at pH 8.0. This suspension was used in baking trials with TAH 4a, Sp.10.1, Sp.10.2, Sp.11.2 and Sp.23.2bis xylanases. In the case of TAH 2a and TAH 7a, however, the resuspended samples were first dialysed overnight against 20 mM MOPS containing 300 mM NaCl and centrifuged at 18,000× g for 1 hour. The soluble (supernatant) and insoluble fractions hence obtained were used in the baking trials for these samples (see example 3).

Characterization of the Enzymes

The activity of the xylanases obtained from strains TAH 2a, TAH 7a (soluble and insoluble fractions as described above) and obtained from strain TAH 4a was determined at various temperatures. The results of this experiment are presented in FIG. 2. The xylanases display maximum activity between 20° C. and 40° C. and retain more than 40% of their activity at 5° C.

### Example 3

### Baking Trials

Baking trials were performed to demonstrate the positive effect of the xylanases obtained in example 2 in baking. The positive effect was evaluated by the increase in bread volume as compared to a commercially available xylanase and as compared to a reference not containing any of these enzymes.

The xylanases of the invention were tested in Belgian hard rolls that are produced on a large scale every day in Belgium. The procedure described is well known to the craft baker and it is obvious to one skilled in the art that the same results may be obtained by using other protocols or equipment from other suppliers.

The ingredients used are listed in table 1 below:

TABLE 1

| Ingredients (g) | |
|---|---|
| Flour (Surbi -Molens van Deinze) | 1500 |
| Water | 915 |
| Fresh yeast (Bruggeman-Belgium) | 90 |
| Sodium chloride | 30 |
| Multec Data HP 20 (Beldem-Belgium) | 3.9 |
| Ascorbic acid | 0.12 |
| Xylanase | See table 2 |

The ingredients were mixed for 2 min at low and 8 min at high speed in a Diosna SP24 mixer. The final dough temperature as well as the resting and proofing temperature was 25° C. After resting for 15 min at 25° C., the dough was reworked manually and rested for another 10 min. Thereafter, 2 kg dough pieces were made up and proofed for 10 min. The 2-kg dough pieces were divided and made up using the Eberhardt Optimat. 66 g round dough pieces were obtained. After another 5 min resting time, the dough pieces were cut by pressing and submitted to a final proofing stage for 70 min. The dough pieces were baked at 230° C. in a MIWE Condo™ oven with steam (Michael Wenz-Arnstein-Germany). The volume of 6 rolls was measured using the commonly used rapeseed displacement method.

The results of the baking trials are presented in table 2 below:

TABLE 2

| | | Rolls volume |
|---|---|---|
| | Xylanase | increase (%) |
| | units/100 kg | compared to control |
| Xylanase sample | flour(*) | without xylanase |
| Belase B210 | 1050 | 16 |
| (Beldem-Belgium) | | |
| TAH 2a soluble(**) | 6000 | 15 |
| TAH 2a insoluble | 6000 | 12 |
| TAH 4a | 6000 | 25 |
| TAH 7a soluble | 6000 | 12 |
| TAH 7a insoluble | 6000 | 14 |
| Sp.10.1. | 6000 | 15 |
| Sp.10.2. | 6000 | 14 |
| Sp.11.2. | 6000 | 13 |
| Sp.23.2.bis. | 6000 | 20 |

(*)One unit of Belase B210 xylanase is defined as the amount of enzyme needed to release 1 μmole of reducing sugar (expressed as xylose) from birchwood xylan at 30° C. and pH 4.5 (Nelson-Somogyi method). One unit of the other xylanases is defined as the amount of enzyme needed to release 1 μmole of reducing sugar (expressed as xylose) from birchwood xylan at 25° C. and pH 6.5 (dinitrosalicylic acid method, Miller, G. 1959, Anal. Chem. Vol. 31, p. 426).
(**)With soluble is meant the (soluble) enzyme fraction present in the supernatant (see above).

The above results show that all the xylanases tested had a positive effect on the volume of bread.

### Example 4

### Cloning of the Xylanase Genes

Bacterial Strains and Culture Conditions

The strains TAH 2a, TAH 4a, TAH 7a, Sp10.1, Sp10.2, Sp11.2, Sp23.2 and Sp23.2bis described in example 1 were cultivated for 16 h in the modified Marine broth described above (see example 2).

Preparation of Genomic DNA

Genomic DNA was extracted and purified from 16 hour cultures, cultivated at 37° C. in the medium described above, with the Wizard® Genomic DNA Purification kit (Promega).

Cloning of the Xylanase Genes

The xylanase genes were PCR-amplified using VENT polymerase (Biolabs) with the sense primer:

SEQ ID NO: 17

5′-GGGCATATGAAAGTATTTTTTAAAATAACAACTT-3′

containing an NdeI site (underlined) and the antisense primer:

SEQ ID NO: 18

5′-GGGCTCGAGTTAATTAAACGTGTTGTTATAAAA-3′

containing an Xho I site (underlined) and the stop codon (in italics). The concentration of the genomic DNA template used was optimized for each source isolate. After 3 min initial denaturation at 95° C., 25 cycles of amplification were performed using a Progene apparatus (Techne Cambridge, UK). Each cycle included denaturation at 95° C. for 1 min, hybridization at 54° C. for 30 sec, and elongation at 72° C. for 1.5 min.

The fragment obtained from each isolate was cloned into a PCRScript Amp SK (+) vector (Stratagene), using the procedure recommended by the supplier, and used to transform XL10-Gold® Kan ultracompetent cells (Stratagene). Blue-white selection allowed selection of white colonies carrying the PCR-fragment. The sequence of the inserted fragment was determined with an ALF DNA sequencer (Pharmacia Biotech) using purified plasmid preparation (Nucleospin plasmid, Macherey-Nagel).

Sequences of the Xylanases Genes

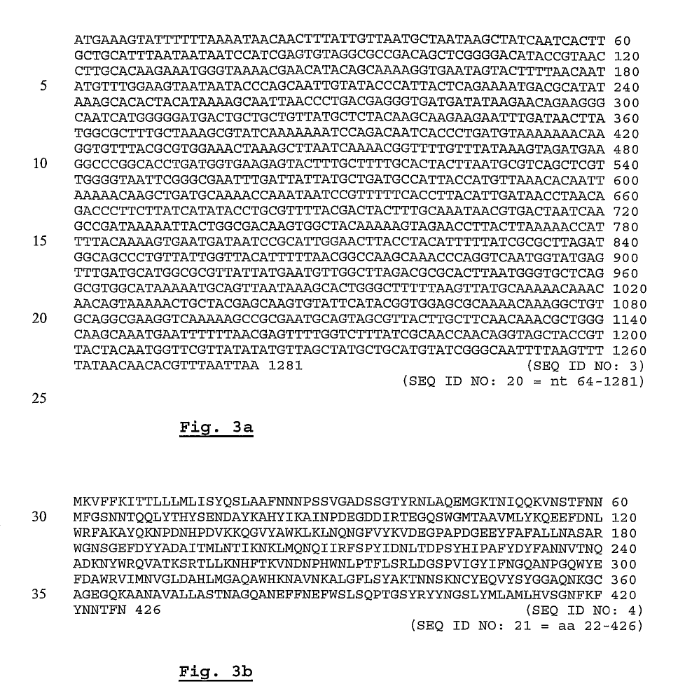

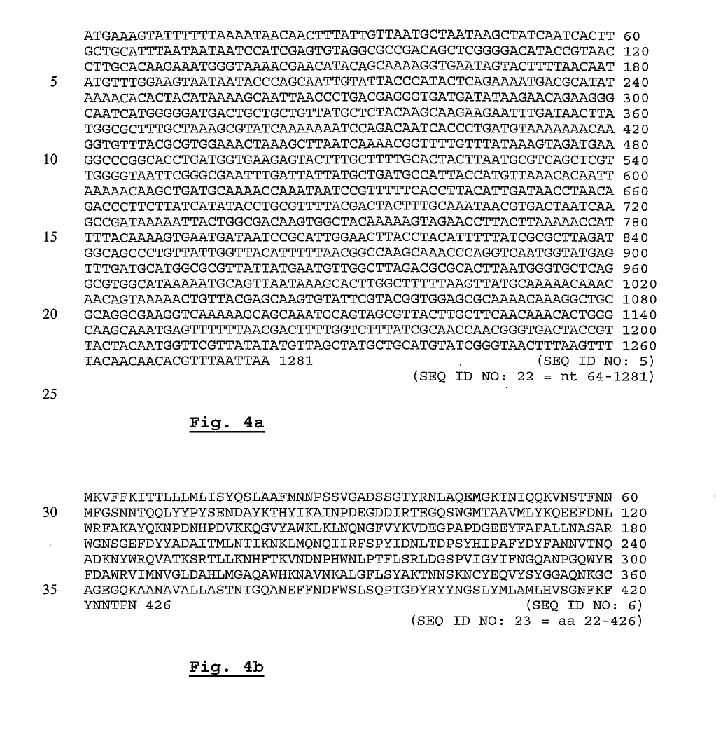

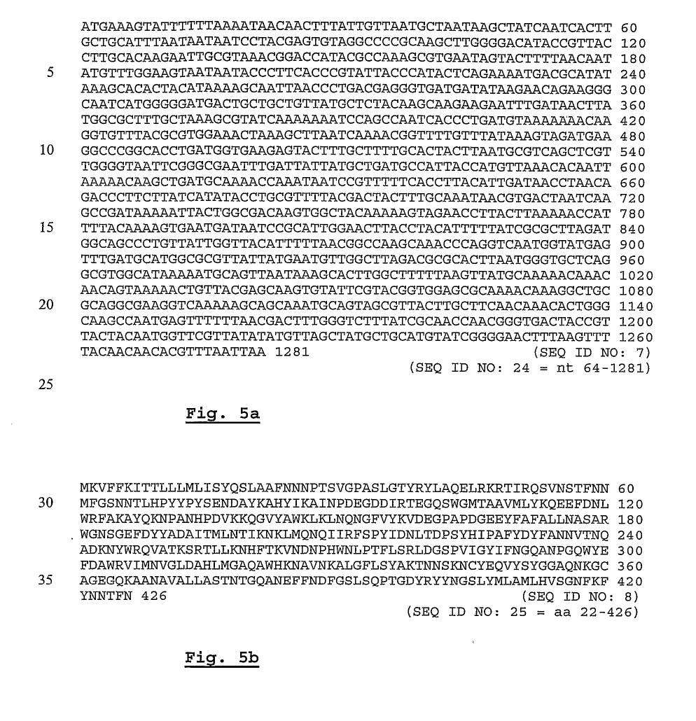

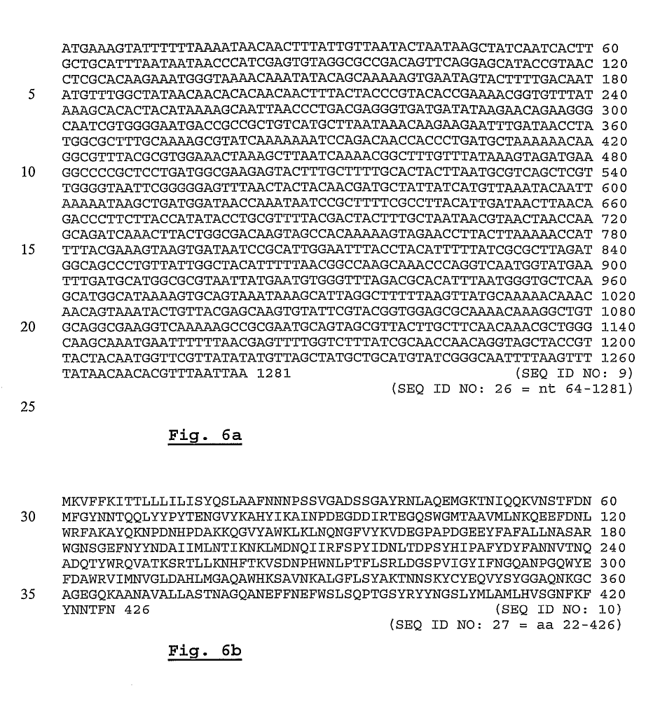

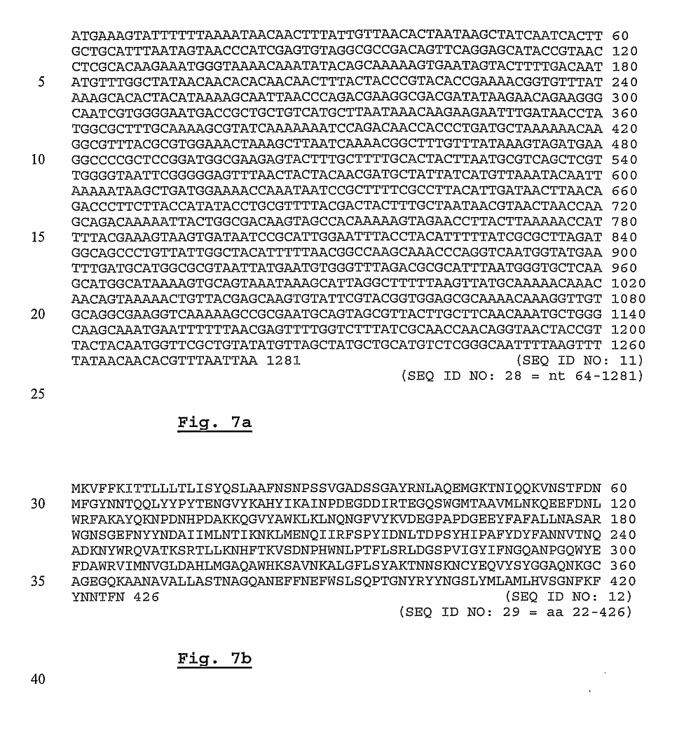

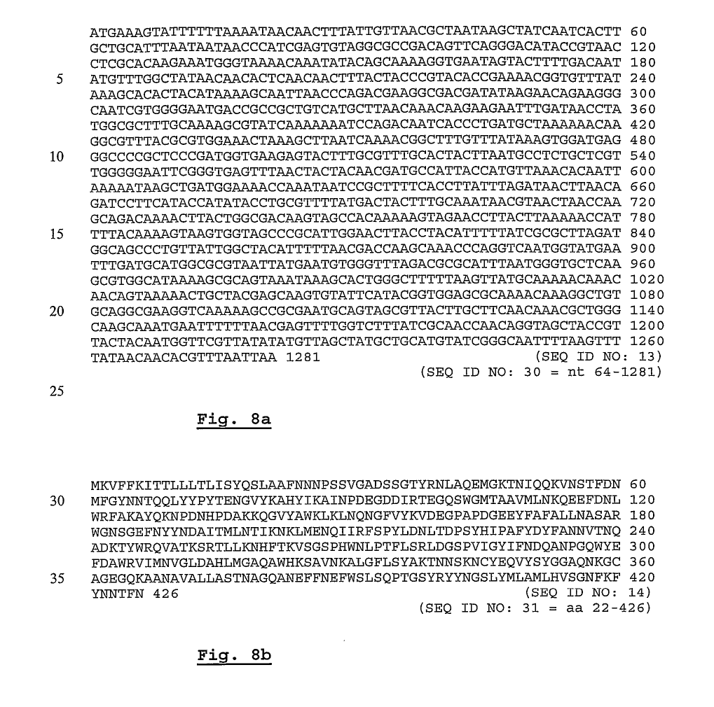

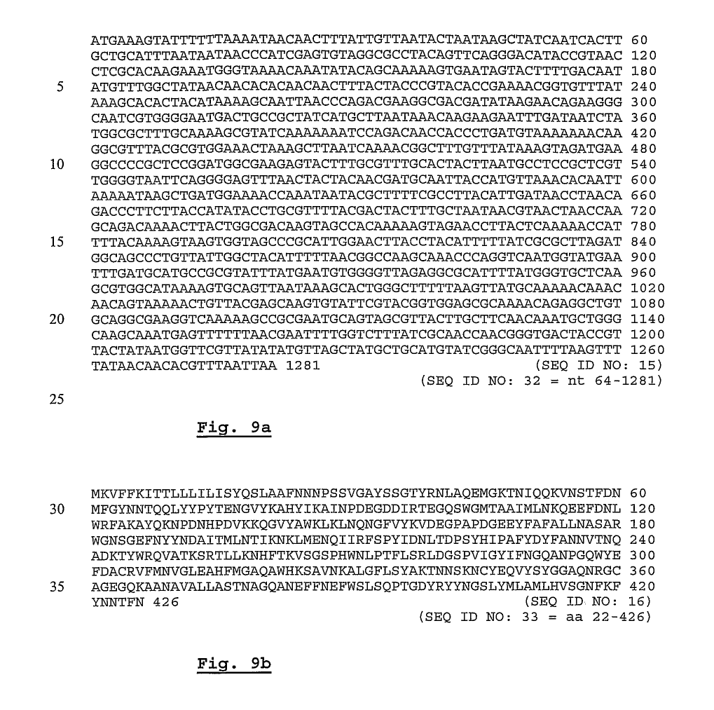

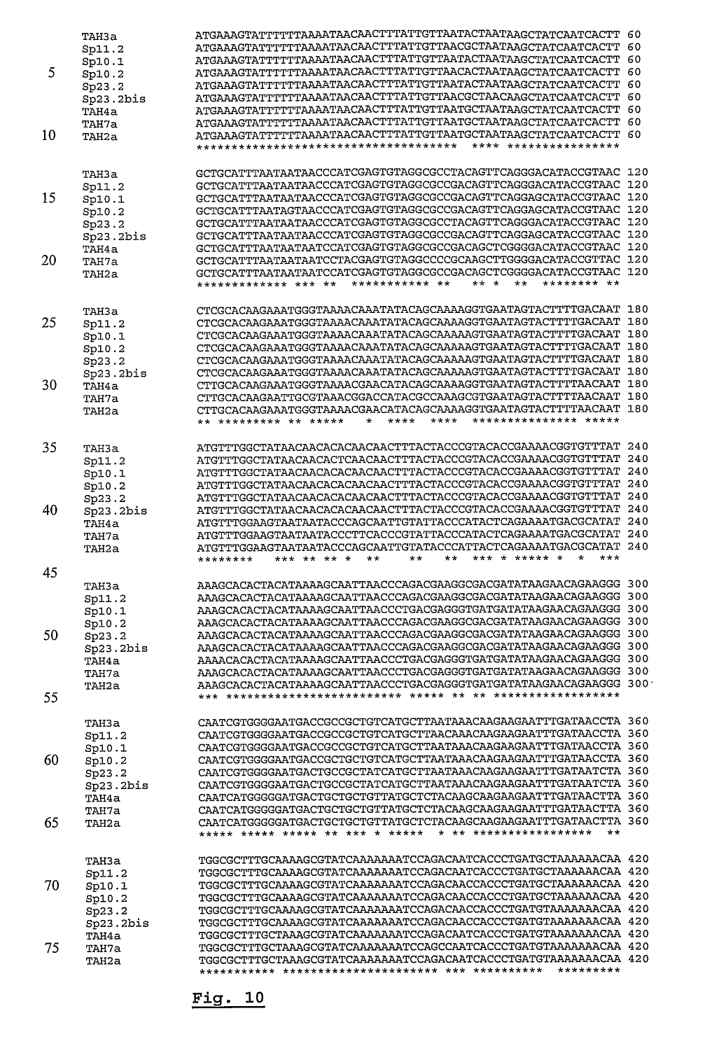

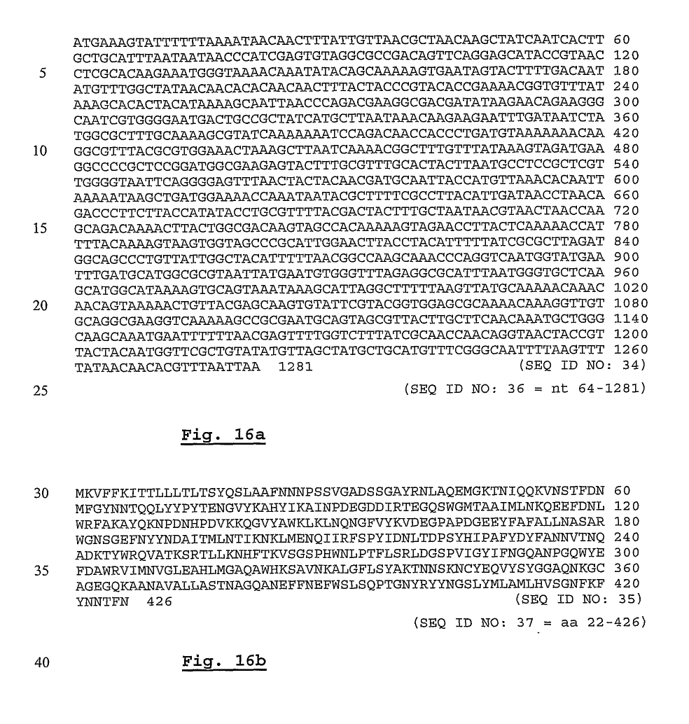

The sequences of the TAH 2a, TAH 4a, TAH 7a, Sp.10.1, Sp.10.2, Sp.11.2, Sp.23.2 and Sp.23.2bis xylanase genes are shown in FIGS. 3-10a and 16a (SEQ ID NOs: 3, 5, 7, 9, 11, 13, 15, 34) and their corresponding amino acid sequences are shown in FIG. 3-10b and 16b (SEQ ID NOs: 4, 6, 8, 10, 12, 14, 16, 35). The sequences encoding for the mature protein portions are given in SEQ ID NOS: 20, 22, 24, 26, 28, 30, 32 and 36.

These sequences are highly similar to each other and are also homologous to the published sequence of the xylanase gene from Pseudoalteromonas haloplanktis TAH 3a (EMBL Accession number AJ427921). All enzymes described in the present invention belong to glycoside hydrolase Family 8.

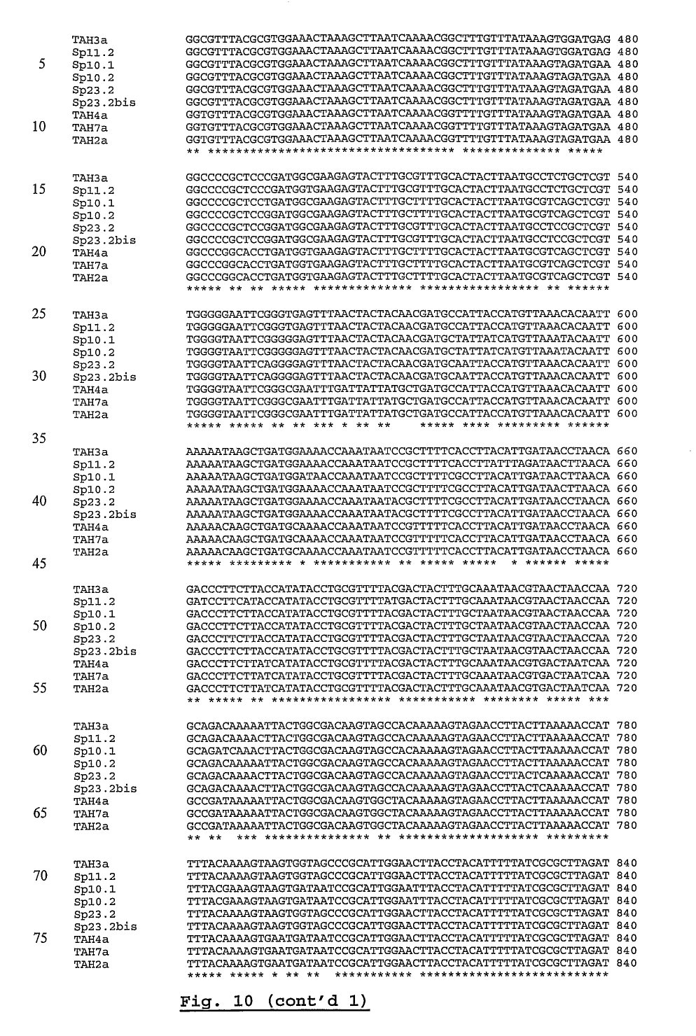

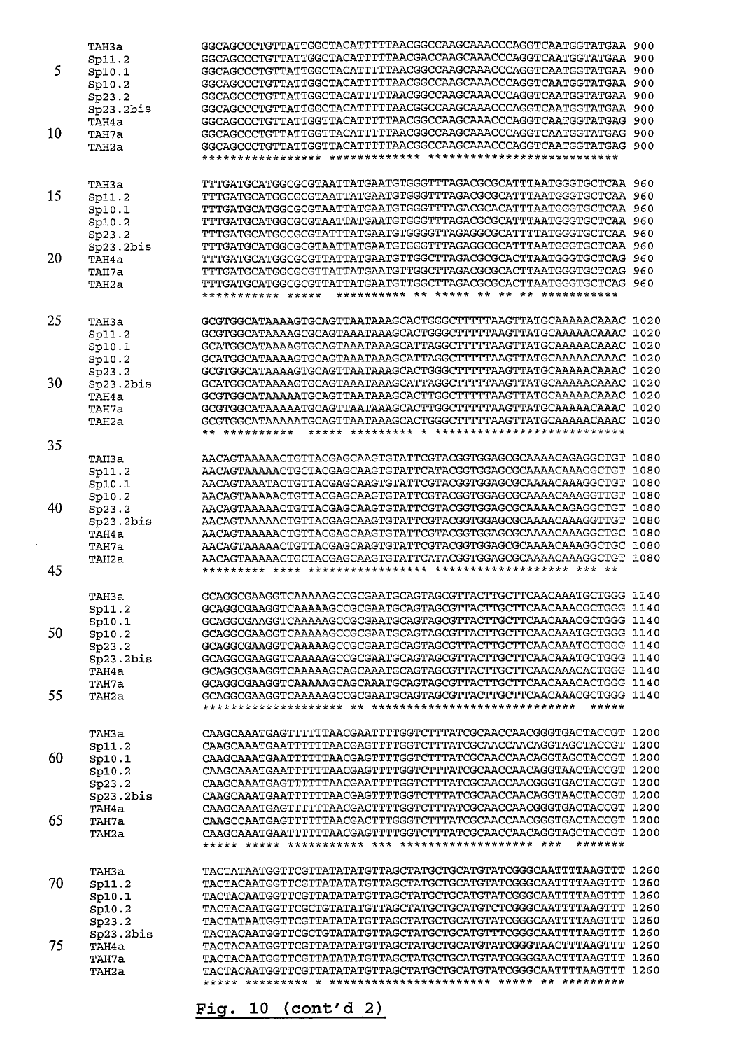

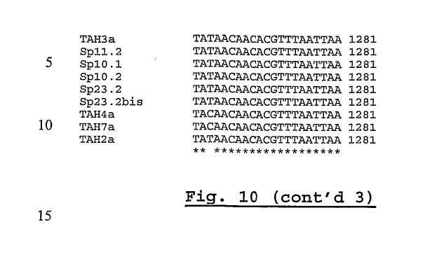

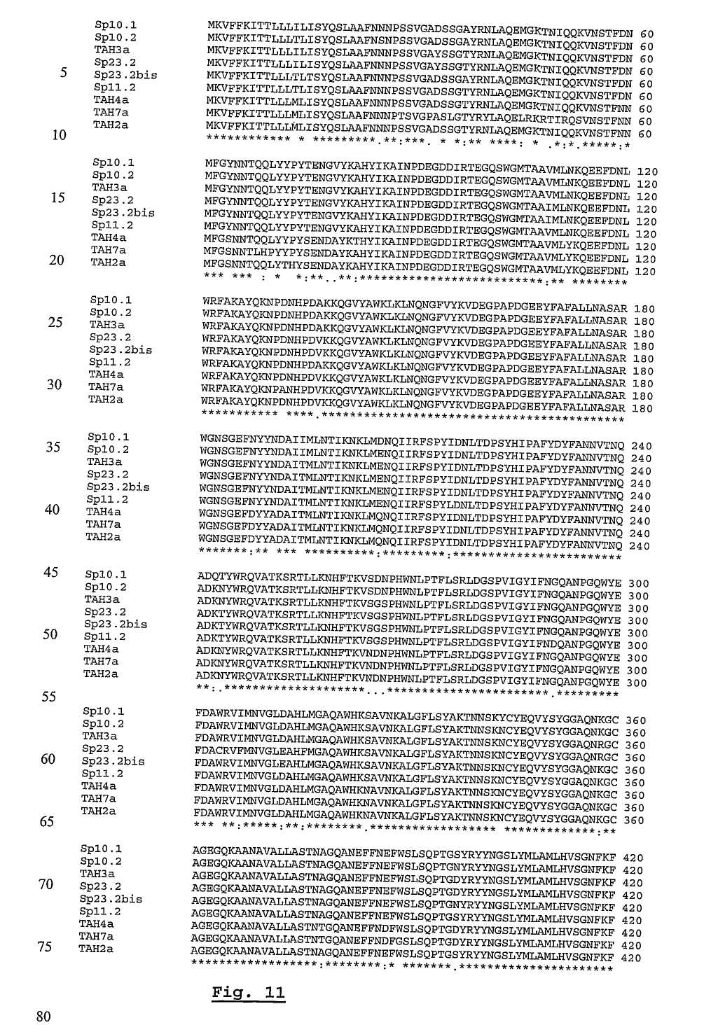

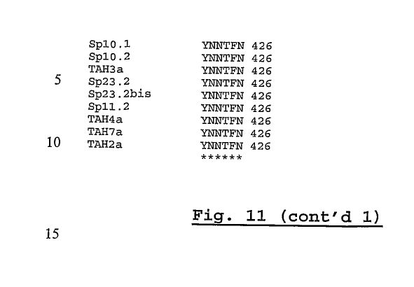

The result of a Clustalw multiple sequence alignment using the default parameters (http://www.ebi.ac.uk/clustalw/) are presented in FIGS. 10 (nucleotide sequences) and 11 (amino acid sequences).

All new sequences differ by at least 20 nucleotides or by at least 4 amino acids from the known published sequence (see below).

In all of the above-identified amino acid sequences, the mature protein (enzyme) starts at amino acid position No. 22.

The closest related amino acid sequences identified by a Blast search using the default parameters (http://www.ncbi.nlm.nih.gov/blast/) are:
1/ the already characterized Family 8 xylanase from Pseudoalteromonas haloplanktis TAH3a (between 92 and 98% sequence identity)
2/ an uncharacterized 1748 amino acids long protein with a C-terminal OMP (outer membrane protein) domain from Cytophaga hutchinsonii (33 to 34% identity on a 412 amino acids span)
3/ xylanase Y from Bacillus halodurans (31 to 32% identity)
4/ xylanase Y from Bacillus sp. KKl (28 to 30% identity)

The amino acid differences between the published Pseudoalteromonas haloplanktis TAH3a xylanase sequence and the xylanase sequences of the present invention have been identified and are listed in Table 3 below. Their respective positions in the structure, as based on the published structure (Van Petegem F. et al., 2003, J. Biol. Chem. Vol. 278, p. 7531) are also shown.

TABLE 3

| TAH 3a | | Mutation | |
|---|---|---|---|
| xylanase | | in the new | |
| residue | | xylanase | Localization in |
| (mature | | (mature | the 3D structure |
| sequence) | New xylanase involved | sequence) | of TAH 3a xylanase |
| Asn 4 | Sp.10.2 | Ser 4 | Loop (N-term) |
| Ser 7 | TAH 7a | Thr 7 | Loop |
| Ala 11 | TAH 7a | Pro 11 | α helix like |
| Ser 14 | TAH 7a | Leu 14 | α helix like |
| Thr 16 | Sp.10.1, Sp.10.2, | Ala 16 | Loop |
| | Sp23.2bis | | |
| Asn 19 | TAH 7a | Tyr 19 | Loop/α helix |
| Met 24 | TAH 7a | Leu 24 | α helix |
| Gly 25 | TAH 7a | Arg 25 | α helix/loop |
| Thr 27 | TAH 7a | Arg 27 | Loop |
| Asn 28 | TAH 7a | Thr 28 | Loop |
| Gln 30 | TAH 7a | Arg 30 | α helix |
| Lys 32 | TAH 7a | Ser 32 | α helix |
| Asp 38 | TAH 2a, TAH 4a, TAH 7a | Asn 38 | α helix |
| Tyr 43 | TAH 2a, TAH 4a, TAH 7a | Ser 43 | Loop (surface) |
| Gln 47 | TAH 7a | Leu 47 | Loop (surface) |
| Gln 48 | TAH 7a | His 48 | Loop (surface) |
| Leu 49 | TAH 7a | Pro 49 | β-sheet (N-term) |
| Tyr 51 | TAH 2a | Thr 51 | β-sheet |
| Thr 54 | TAH 2a, TAH 4a, TAH 7a | Ser 54 | B-sheet |
| Gly 57 | TAH 2a, TAH 4a, TAH 7a | Asp 57 | Loop |
| Val 58 | TAH 2a, TAH 4a, TAH 7a | Ala 58 | β-sheet (N-term) |
| Ala 61 | TAH 4a | Thr 61 | β-sheet |
| Val 88 | Sp.23.2, Sp23.2bis | Ile 88 | α helix |
| Asn 91 | TAH 2a, TAH 4a, TAH 7a | Tyr 91 | Loop |
| Asp 111 | TAH 7a | Ala 111 | Loop (surface) |
| Ala 116 | TAH 2a, TAH 4a, TAH 7a, | Val 116 | Loop (surface) |
| | Sp.23.2, sp23.2bis | | |
| Asn 167 | TAH 2a, TAH 4a, TAH 7a | Asp 167 | α helix/loop |
| Asn 170 | TAH2a, TAH4a, TAH7a | Ala 170 | α helix |
| Thr 174 | Sp.10.1, Sp.10.2 | Ile 174 | α helix |
| Glu 185 | Sp.10.1 | Asp 185 | β-sheet (C-term) |
| Glu 185 | TAH 2a, TAH 4a, TAH 7a | Gln 185 | β-sheet (C-term) |
| Ile 195 | Sp.11.2 | Leu 195 | Loop |
| Lys 222 | Sp.10.1 | Gln 222 | α helix |
| Asn 223 | Sp.23.2, Sp.11.2, | Thr 223 | α helix |
| | Sp.10.1, sp23.2bis | | |
| Ser 244 | TAH 2a, TAH 4a, TAH 7a | Asn 244 | Loop |
| Gly 245 | Sp.10.1, Sp.10.2, | Asp 245 | Loop |
| | TAH 2a, TAH 4a, TAH 7a | | |
| Ser 246 | Sp.10.1, Sp.10.2, | Asn 246 | Loop |
| | TAH 2a, TAH 4a, TAH 7a | | |
| Gly 270 | Sp.11.2 | Asp 270 | Loop (near the |
| | | | active site) |
| Trp 283 | Sp.23.2 | Cys 283 | α helix (near the |
| | | | active site) |
| Ile 286 | Sp.23.2 | Phe 286 | α helix |
| Asp 292 | Sp.23.2, Sp23.2bis | Glu 292 | α helix |
| Leu 295 | Sp.23.2 | Phe 295 | α helix (C-term) |
| Ser 304 | TAH 2a, TAH 4a, TAH 7a | Asn 304 | α helix |
| Asn 323 | Sp.10.1 | Tyr 323 | Loop (surface) |
| Arg 337 | Sp.11.2, Sp.10.1, | Lys 337 | Loop |
| | Sp.10.2, TAH 2a, | | |
| | TAH 4a, TAH 7a, | | |
| | Sp23.2bis | | |
| Ala 358 | TAH 4a, TAH 7a | Thr 358 | α helix (N-term) |
| Glu 367 | TAH 4a, TAH 7a | Asp 367 | α helix |
| Asp 377 | Sp.11.2, Sp.10.1, | Ser 377 | Loop |
| | TAH 2a | | |
| Asp 377 | Sp.10.2, Sp23.2bis | Asn 377 | Loop |

### Example 5

### Purification of the Xylanases

Overexpression of the Isolated Xylanases

The xylanase gene fragments were excised from PCRScript Amp SK(+) (see example 4 above) with Nde I and Xho I and ligated into the pET 22b(+) expression vector (Novagen). The resulting recombinant plasmids were transformed to E. coli BL21 (DE3) cells (Stratagene).

Plasmids pXYP4 to pXYP10 were transformed to Escherichia coli JM109 (LMBP 4860 to LMBP 4866).

The plasmid containing the xylanase gene from strain TAH 4a was labeled pXYP4 (see LMBP 4860).

The plasmid containing the xylanase gene from strain TAH 7a was labeled pXYP5 (see LMBP 4861).

The plasmid containing the xylanase gene from strain TAH 2a was labeled pXYP6 (see LMBP 4862).

The plasmid containing the xylanase gene from strain Sp.10.1 was labeled pXYP7 (see LMBP 4863).

The plasmid containing the xylanase gene from strain Sp.10.2 was labeled pXYP8 (see LMBP 4864).

The plasmid containing the xylanase gene from strain Sp.11.2 was labeled pXYP9 (see LMBP 4865).

The plasmid containing the xylanase gene from strain Sp.23.2bis was labeled pXYP10 (see LMBP 4866).

Purification of the Xylanases

The xylanases from the TAH 2a, TAH 4a, TAH 7a, Sp10.1, Sp10.2, Sp11.2, Sp23.2 and Sp23.2bis strains were purified as described (Collins et al, 2002, see above) from liquid cultures of recombinant E. coli BL21 (DE3) strains. Five ml of an overnight preculture (18° C.) of the E. coli recombinant strain, expressing one of the cloned genes of example 2, was centrifuged at 10,000× g for 1 min and the pellet was resuspended in 300 ml of Terrific broth (12g/l Bacto tryptone (Difco), 24 g/l yeast extract (Difco), 4 ml/l glycerol, 12.54 g/l K₂HPO₄, 2.31 g/l KH₂PO₄) containing 200 μg/ml ampicillin in a 3-liter shake flask. The culture was incubated at 18° C. and 250 rpm until an absorbance at 550 nm of between 3 and 4 was reached, whereupon the expression of the enzyme was induced with 1 mM isopropyl-l-thio-β-galactopyranoside.

Following a further 20 hours incubation at 18° C., the cells were harvested by centrifugation at 18,000× g for 30 min at 4° C., resuspended in buffer A (50 mM BICINE, pH 8.5), disrupted in a prechilled cell disrupter (Constant Systems Ltd.) at 28 Kpounds/square inch, centrifuged at 40.000× g, and dialyzed against buffer A. The dialysate was loaded on a Q-Sepharose Fast flow (Amersham Biosciences) column (50×2.5 cm) equilibrated in the same buffer, and the void was collected. In the case of the xylanases from TAH 2a (SEQ ID NO: 4), TAH 4a (SEQ ID NO: 6), TAH 7a (SEQ ID NO: 8), Sp.11.2 (SEQ ID NO: 14), Sp.23.2 (SEQ ID NO: 16) and Sp.23.2bis (SEQ ID NO: 35), this solution was immediately loaded on a S-Sepharose Fast flow (Amersham Biosciences) column (50×2.5 cm), also equilibrated in the above mentioned buffer, while in the case of the xylanase from Sp10.1 (SEQ ID NO: 10) and Sp10.2 (SEQ ID NO: 12) the pH of this solution was first adjusted to pH 7.6 before loading on a S-Sepharose Fast flow (Amersham Biosciences) column (50×2.5 cm) equilibrated in 50 mM BICINE, pH7.6. Bound protein was eluted with a linear NaCl gradient (0-100 mM in 350 ml) and active fractions were pooled and concentrated by ultrafiltration (Millipore PBGC 10,000 NMWL). This solution was used in the baking trials with xylanases from the strains Sp.10.1 and Sp.10.2, while the xylanases from TAH 2a, TAH 4a, TAH 7a, Sp.11.2, SP.23.2 and Sp.23.2bis strains were firstly further purified on a Sephacryl S-100 (Amersham Biosciences) column (90×2.5 cm) equilibrated in 20 mM MOPS, 100 mM NaCl, pH 7.5 at 1 ml/min.

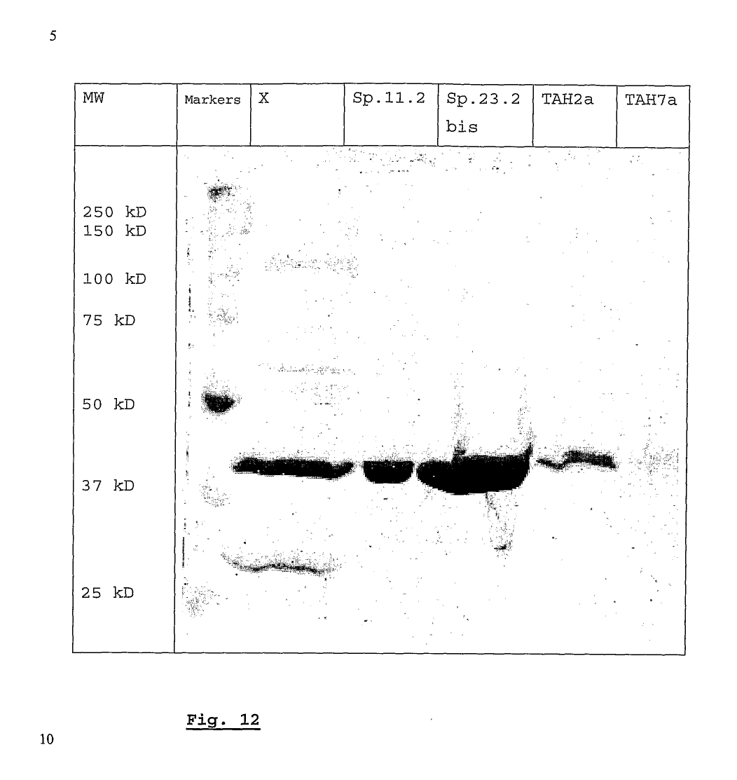

The purity of the enzymes was demonstrated by electrophoresis on a SDS-polyacrylamide gel. The results thereof are presented in FIG. 12.

### Example 6

### Effect of the Recombinant Xylanases in Minibaking Test

Baking trials were performed to demonstrate similar positive effect of the recombinant xylanases of the present invention in baking. The positive effect was evaluated by the increase in bread volume as compared to a commercially available xylanase and as compared to a reference not containing any of these enzymes.

The purified or partially purified xylanases from example 5 were evaluated in mini baking tests consisting of preparing dough with 100 g of flour.

The procedure described is well known to one skilled in the art and it is obvious that the same results may be obtained by using other protocols or equipment from other suppliers.

The ingredients used are listed in table 4 below:

TABLE 4

| Ingredients (g) | RECIPE |
|---|---|
| Flour (Surbi -Molens van Deinze) | 100 |
| Water | 57 |
| Fresh yeast (Bruggeman-Belgium) | 5 |
| Sodium chloride | 2 |
| Dextrose | 2 |
| Ascorbic acid (g/100 kg flour) | 4 |
| Xylanase | see Table 5 |

The ingredients were mixed for 4.5 min in a National mixer. 150 g dough pieces were weighed and rested for 20 min at 25° C. in plastic boxes. The doughs were reworked and rested for a further 20 min. The final proofing time was 50 min at 36° C. The dough pieces were then baked at 225° C. for 20 min. The volume of the bread was measured using the commonly used rapeseed displacement method.

The results of the baking trials with the recombinant enzymes are presented in table 5 below:

TABLE 5

| | | Rolls volume increase |
|---|---|---|
| | Xylanase | (%) compared to |
| | units/100 | control without |
| Xylanase sample | kg flour(*) | xylanase |
| Belase B210 | 1050 | 18 (¹) |
| (Beldem-Belgium) | | |
| TAH 2a | 6000 | 23 (²) |
| TAH 4a | 6000 | 21 |
| TAH 7a | 6000 | 23 |
| Sp.10.1. | 6000 | 27 |
| Sp.10.2. | 6000 | 21 |
| Sp.11.2. | 6000 | 20 |
| Sp.23.2.bis. | 6000 | 17 |

(*)One unit of Belase B210 xylanase is defined as the amount of enzyme needed to release 1 μmole of reducing sugar (expressed as xylose) from birchwood xylan at 30° C. and pH 4.5 (Nelson-Somogyi method). One unit of the other xylanases is defined as the amount of enzyme needed to release 1 μmole of reducing sugar (expressed as xylose) from birchwood xylan at 25° C. and pH 6.5 (dinitrosalicylic acid method, Miller, G. 1959, Anal. Chem. Vol. 31, p. 426).
(¹) mean value of three independent trials
(²) mean value of two independent trials

The above results show that the xylanases produced by a recombinant host cell have a similar positive effect on the volume of bread as compared to the wild type enzymes.

### Example 7

### Volume-Response Test for Family 8 Xylanases used in Baking

Baking trials were performed to demonstrate similar positive effect of the recombinant xylanases of the present invention in baking and to determine the effect of the amounts of xylanase added. The positive effect was evaluated by the increase in bread volume.

The results of the baking trials, with the recombinant enzymes are presented in table 6 below. The protocol as described in Example 3 was followed:

TABLE 6
Loaf volume in function of units xylanase added

| DXU/100 kg | SP11.2 | SP10.2 | TaH3a | TaH2a | SP23.2.bis |
|---|---|---|---|---|---|
| of flour(*) | (×1000) | (×1000) | (×1000) | (×1000) | (×1000) |
| 0 | 1350 | 1350 | 1350 | 1350 | 1350 |
| 685 | 1525 | | | | |
| 960 | | | | 1575 | |
| 1370 | 1600 | | | | |
| 1410 | | 1600 | | | |
| 1465 | | | | | 1575 |
| 1500 | | | 1625 | | |
| 1920 | | | | 1600 | |
| 2055 | 1600 | | | | |
| 2150 | | | 1675 | | |
| 2740 | 1625 | | | | |
| 2820 | | 1675 | | | |
| 2880 | | | | 1625 | |
| 2930 | | | | | 1575 |
| 3000 | 1650 | | | | |
| 3840 | | | | 1675 | |
| 4230 | | 1650 | | | |
| 4300 | | | 1700 | | |
| 4395 | | | | | 1575 |
| 4500 | 1750 | | | 1750 | |
| 5000 | | | | | |
| 5640 | | 1750 | | | |
| 5860 | | | | | 1650 |
| 6000 | 1725 | | | 1850 | |
| 6450 | | | 1700 | | |
| 8600 | | | 1725 | | |

(*)One unit of the other xylanases is defined as the amount of enzyme needed to release 1 μmole of reducing sugar (expressed as xylose) from birchwood xylan at 25° C. and pH 6.5 (dinitrosalicylic acid method, Miller, G. 1959, Anal. Chem. Vol. 31, p. 426).

The results are graphically represented in FIG. 15.

### Example 8

### Absolute Amounts of Xylanase used in Baking

The relative amounts of the xylanases described in the above examples, needed to obtain a defined effect on the volume in the Belgian hard rolls assay, have been compared to a commercial commonly used bacterial xylanase (Bel'ase B210—Beldem—Belgium).

The protein concentration of the purified xylanases was determined by absorbance measurement at 280nm using extinction coefficients of 92,860 (in the case of TAH 2a), 94,140 (in the case of TAH 4a and Sp.11.2), 89,730 (in the case of TAH 7a and Sp.23.2), 95,420 (in the case of Sp.10.1) and 94,140 (in the case of Sp.10.2) M⁻cm⁻¹, and/or with the Bradford protein assay kit (Pierce) as described by the manufacturers.

Several baking trials were performed, according to the method described in example 6, with various amounts of the purified enzymes as described in example 5.

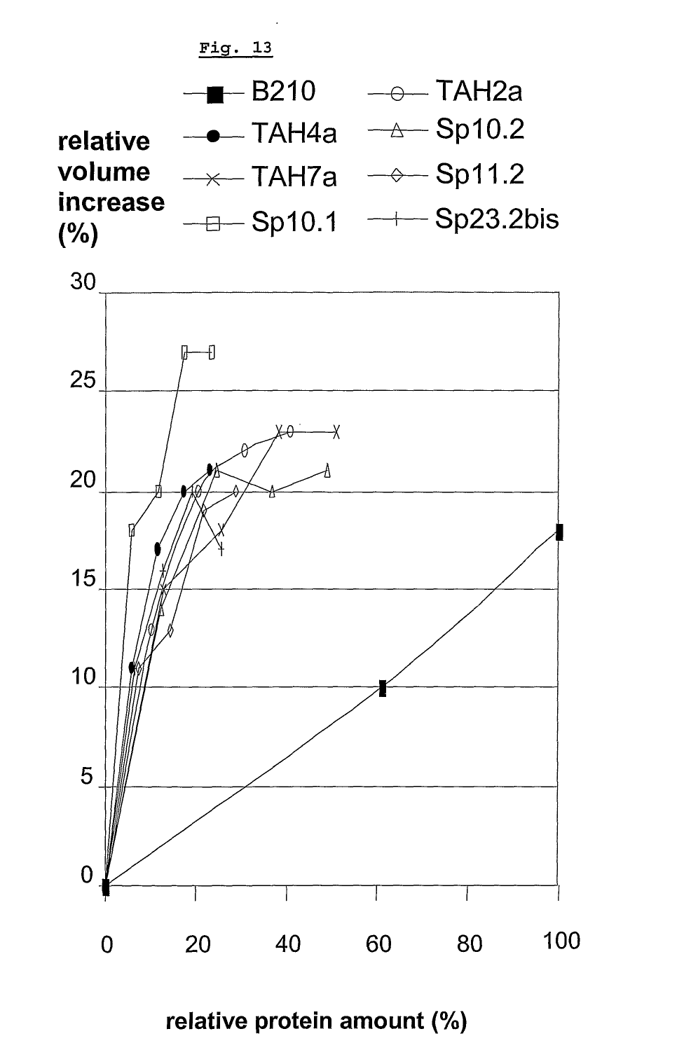

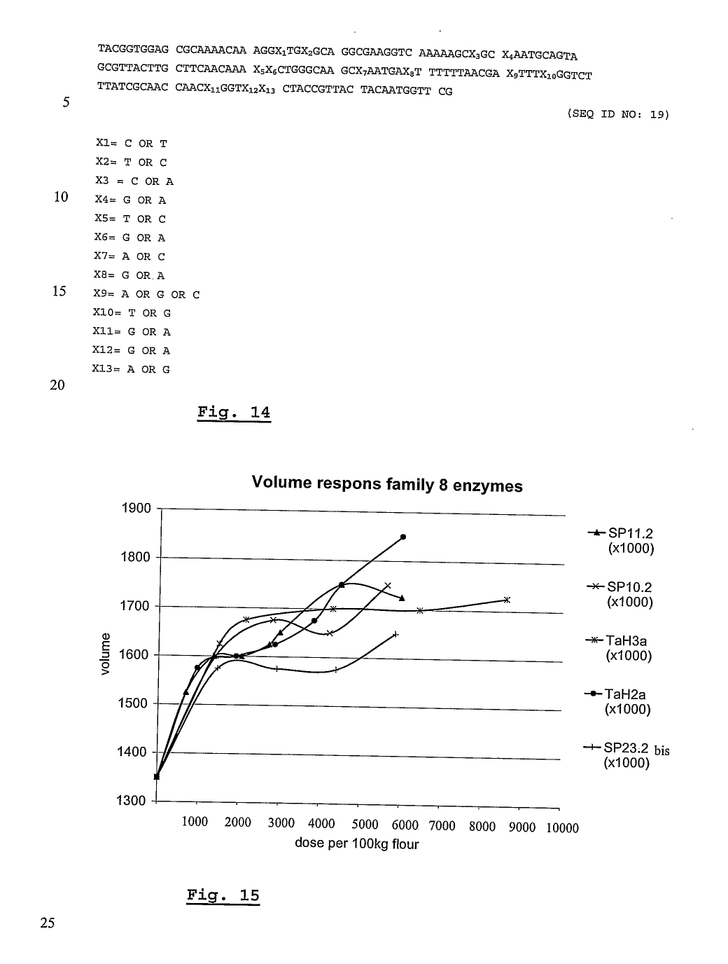

FIG. 13 shows the increase of the bread volume as a function of the relative quantity of the different enzymes. For the Bel'ase B210 xylanase (about half the size of the enzymes of the invention), the results are the mean values of three independent trials. For the TAH2a xylanase, the results are the mean values of two independent trials.

FIG. 13 clearly shows that the specific “in pano” activity (i.e. volume of bread expressed in ml/protein amount expressed in mg) is significantly higher for the xylanases of the present invention as compared to the commercial enzyme.

The applicant has made on 4 march 2004 a deposit (under the expert solution) of the micro-organisms Escherichia coli JM109 (pXYP4), Escherichia coli JM109 (pXYP5), Escherichia coli JM109 (pXYP6), Escherichia coli JM109 (pXYP7), Escherichia coli JM109 (pXYP8), Escherichia coli JM109 (pXYP9), Escherichia coli JMlO9 (pXYP10) according to the invention, at the BCCM/LMBP culture collection (Laboratorium voor Moleculaire Biologie, Universiteit Ghent, ‘Fiers-Schell-Van Montagu’ building, Technologiepark 927, B-9052 Gent-Zwijnaarde, Belgium). These deposits received accession numbers LMBP 4860, LMBP 4861, LMBP 4862, LMBP 4863, LMBP 4864, LMBP 4865 and LMBP 4866 respectively.

1. An isolated and purified enzyme with xylanolytic activity belonging to glycoside hydrolase Family 8, wherein the enzyme is a psychrophilic enzyme, with the proviso that said xylanolytic enzyme is not encoded by SEQ ID NO: 1, and comprising an amino acid sequence corresponding to
aa 1 to aa 426 of any of SEQ ID NOs: 4, 6, 8, 10, 12, 14, 16 or 35;
aa 22 to aa 426 of any of SEQ ID NOs: 4, 6, 8, 10, 12, 14, 16 or 35;
or a variant thereof comprising an amino acid sequence that differs in less than 4 amino acids from any of the above sequences.

2. The enzyme according to claim 1, wherein said enzyme increases the volume of a baked product by at least 10% and/or increases the width of cut of said baked product when added to a dough in a concentration of between 1500 and 6000 xylanase units/100 kg flour.

3. An isolated and purified enzyme with xylanolytic activity according to claim 1, consisting of an amino acid sequence corresponding to
aa 1 to aa 426 of any of SEQ ID NOs: 4, 6, 8, 10, 12, 14, 16 or 35;
aa 22 to aa 426 of any of SEQ ID NOs: 4, 6, 8, 10, 12, 14, 16 or 35;
or a variant thereof consisting of an amino acid sequence that differs in less than 4 amino acids from any of the above sequences.

4. The enzyme according to claim 1 which is obtained from a bacterial strain with deposit number LMBP 4860, LNBP 4861, LMBP 4862, LMBP 4863, LMBP 4864, LMBP 4865 or LMBP 4866.

5. An isolated and purified nucleotide sequence encoding an enzyme according to claim 1.

6. The nucleotide sequence according to claim 5 comprising SEQ ID NO: 19.

7. The nucleotide sequence according to claim 5 comprising a nucleotide sequence corresponding to
nt 1 to nt 1281 of any of SEQ ID NOs: 3, 5, 7, 9, 11, 13, 15 or 34;
nt 64 to nt 1281 of any of SEQ ID NOs: 3, 5, 7, 9, 11, 13, 15 or 34;
or a variant of any of the above having a nucleotide sequence that differs in less than 20 nucleotides from any of the above sequences.

8. The nucleotide sequence according to claim 5 consisting of a nucleotide sequence corresponding to
nt 1 to nt 1281 of any of SEQ ID NOs: 3, 5, 7, 9, 11, 13, 15 or 34;
nt 64 to nt 1281 of any of SEQ ID NOs: 3, 5, 7, 9, 11, 13, 15 or 34; or
a variant of any of the above having a nucleotide sequence that differs in less than nucleotides from any of the above sequences.

9. A recombinant nucleotide sequence comprising, operably linked to one or more nucleotide sequences according to claim 5.

10. A vector comprising a nucleotide sequence according to 5.

11. The vector according to claim 10, being a plasmid incorporated in Escherichia coli and having a deposit number selected from the group consisting of LMBP 4860, LMBP 4861, LMBP 4862, LMBP 4863, LMBP 4864, LMBP 4865 and LMBP 4866.

12. A recombinant host cell transformed with a nucleotide sequence of claim 5 or with a vector according to claim 10.

13. The recombinant host cell according to claim 12, selected from the group consisting of bacteria, fungi, and yeast.

14. The host cell of claim 12 extra-cellularly expressing an enzyme according to claim 1.

15. The host cell of claim 12 intra-cellularly expressing an enzyme according to claim 1.

16. A solid support having fixed thereto at least one element selected from the group consisting of a recombinant host cell according to claim 12 a cell extract of the said cell, said cell extract comprising an enzyme with xylanolytic activity according to claim 1, and an isolated and purified enzyme with xylanolytic activity according to claim 1.

17. A bread improving composition comprising at least one of the enzymes according to claim 1.

18. The bread improving composition according to claim 17 further comprising at least one bread improving agent selected from the group consisting of enzymes, emulsifiers, oxidants, milk powder, fats, sugars, amino acids, salts and proteins.

19. The bread improving composition according to claim 18, wherein said enzyme is selected from the list consisting of alpha-amylases, beta-amylases, maltogenic amylases, xylanases, proteases, glucose oxidase, oxido-reductases, glucanases, cellulases, transglutaminases, isomerases, lipases, phospholipases, and pectinases.

20. The bread improving composition of claim 19, wherein said alpha-amylase is an alpha-amylase obtained from Aspergillus oryzae.

21. A method for the degradation of plant cell wall components said method comprising adding an enzyme of claim 1 or a host cell of claim 12 to a composition or material comprising plant cell wall components to be degraded.

22. The method according to claim 21 wherein the composition or material is selected from the group consisting of plants, fruits, legume juice, beer, paper, starch, gluten and vegetable oil.

23. The method according to claim 21, comprising adding said enzyme or said host cell in the course of a process selected from the group consisting of in fruit, vegetable and plant processing, wine making, brewing, coffee processing, processing of paper or textile, bioconversion processes, processes for decomposing wastes, preferably for decomposing agricultural wastes or wastes from paper mills, vegetable oil preparation process, starch-gluten separation processes, feed preparation, baking, milling , and pastry or confection.

24. The method of claim 23, wherein the process is a baking, milling, pastry or confectionery process, and wherein said adding of the enzyme or the host cell results in increasing the volume of baked products and/or increasing the width of cut on the surface of said baked products.

25. (canceled)

26. The method according to claim 23 wherein said enzyme is combined with one or more enzymes is selected from the group consisting of alpha-amylases, beta-amylases, maltogenic amylases, xylanases, proteases, glucose oxidase, oxido-reductases, glucanases, cellulases, transglutaminases, isomerases, lipases, phospholipases, and pectinases.

27. The method according to claim 26 wherein said host cell or said enzyme is incorporated in a bread improving composition.

28. The method according to claim 27, further comprising adding the bread improving composition is during mixing of a dough, preferably in a concentration of between 1500 and 6000 xylanase units/100 kg flour.

29. The method according to claim 21, wherein said enzyme with xylanolytic activity is added as a cell extract, a cell-free extract or is used as a purified protein.

30. The method according to claim 21, wherein said enzyme with xylanolytic activity is added in the form of a dry powder, a granulate, preferably a non-dusting granulate, or in the form of a liquid.

31. A composition comprising at least one enzyme according to claim 1 and an enz yme having an amino acid sequence according to SEQ ID NO: 2 or the mature portion thereof.

32. The composition of claim 31, comprising at least two enzymes according to claim 1.

33. The enzyme with xylanolytic activity of claim 1, comprising an amino acid sequence that has more than 70% sequence identity with the sequence(s) of claim 1.

34. The enzyme with xylanolytic activity of claim 1, comprising an amino acid sequence that has more than 90% sequence identity with the sequence(s) of claim 1.

35. The enzyme with xylanolytic activity of claim 1, comprising an amino acid sequence that has more than 95% sequence identity with the sequence(s) of claim 1.

36. The enzyme with xylanolytic activity of claim 1, comprising an amino acid sequence that has more than 99% sequence identity with the sequence(s) of claim 1.

37. The enzyme with xylanolytic activity of claim 1, consisting of an amino acid sequence that has more than 70% sequence identity with the sequence(s) of claim 1.

38. The enzyme with xylanolytic activity of claim 1, consisting of an amino acid sequence that has more than 90% sequence identity with the sequence(s) of claim 1.

39. The enzyme with xylanolytic activity of claim 1, consisting of an amino acid sequence that has more than 95% sequence identity with the sequence(s) of claim 1.

40. The enzyme with xylanolytic activity of claim 1, consisting of an amino acid sequence that has more than 99% sequence identity with the sequence(s) of claim 1.

41. The composition of claim 18, wherein said proteins are selected from the group consisting of gluten and proteins having cellulose binding sites.

42. The method of claim 30, wherein the enzyme is mixed with one or more stabilizers selected from the group consisting of polyols, sugars, organic acids and sugar alcohols.


SEQUENCE LISTING (37 sequences)

SEQ ID NO: 1 - DNA, 1281 bp, Pseudoalteromonas haloplanktis
  misc_feature: Xylanase sequence obtained from microbial strain TAH 3a
  CDS (1)..(1281)
atgaaagtattttttaaaataacaactttattgttaatactaataagctatcaatcacttgctgcatttaataataacccatcgagtgtaggcgcctacagttcagggacataccgtaacctcgcacaagaaatgggtaaaacaaatatacagcaaaaggtgaatagtacttttgacaatatgtttggctataacaacacacaacaactttactacccgtacaccgaaaacggtgtttataaagcacactacataaaagcaattaacccagacgaaggcgacgatataagaacagaagggcaatcgtggggaatgaccgccgctgtcatgcttaataaacaagaagaatttgataacctatggcgctttgcaaaagcgtatcaaaaaaatccagacaatcaccctgatgctaaaaaacaaggcgtttacgcgtggaaactaaagcttaatcaaaacggctttgtttataaagtggatgagggccccgctcccgatggcgaagagtactttgcgtttgcactacttaatgcctctgctcgttgggggaattcgggtgagtttaactactacaacgatgccattaccatgttaaacacaattaaaaataagctgatggaaaaccaaataatccgcttttcaccttacattgataacctaacagacccttcttaccatatacctgcgttttacgactactttgcaaataacgtaactaaccaagcagacaaaaattactggcgacaagtagccacaaaaagtagaaccttacttaaaaaccattttacaaaagtaagtggtagcccgcattggaacttacctacatttttatcgcgcttagatggcagccctgttattggctacatttttaacggccaagcaaacccaggtcaatggtatgaatttgatgcatggcgcgtaattatgaatgtgggtttagacgcgcatttaatgggtgctcaagcgtggcataaaagtgcagttaataaagcactgggctttttaagttatgcaaaaacaaacaacagtaaaaactgttacgagcaagtgtattcgtacggtggagcgcaaaacagaggctgtgcaggcgaaggtcaaaaagccgcgaatgcagtagcgttacttgcttcaacaaatgctgggcaagcaaatgagttttttaacgaattttggtctttatcgcaaccaacgggtgactaccgttactataatggttcgttatatatgttagctatgctgcatgtatcgggcaattttaagttttataacaacacgtttaattaa

SEQ ID NO: 2 - protein, 426 aa, Pseudoalteromonas haloplanktis
MKVFFKITTLLLILISYQSLAAFNNNPSSVGAYSSGTYRNLAQEMGKTNIQQKVNSTFDNMFGYNNTQQLYYPYTENGVYKAHYIKAINPDEGDDIRTEGQSWGMTAAVMLNKQEEFDNLWRFAKAYQKNPDNHPDAKKQGVYAWKLKLNQNGFVYKVDEGPAPDGEEYFAFALLNASARWGNSGEFNYYNDAITMLNTIKNKLMENQIIRFSPYIDNLTDPSYHIPAFYDYFANNVTNQADKNYWRQVATKSRTLLKNHFTKVSGSPHWNLPTFLSRLDGSPVIGYIFNGQANPGQWYEFDAWRVIMNVGLDAHLMGAQAWHKSAVNKALGFLSYAKTNNSKNCYEQVYSYGGAQNRGCAGEGQKAANAVALLASTNAGQANEFFNEFWSLSQPTGDYRYYNGSLYMLAMLHVSGNFKFYNNTFN

SEQ ID NO: 3 - DNA, 1281 bp, Pseudoalteromonas sp.
  misc_feature: Xylanase sequence obtained from microbial strain TAH 2a
  CDS (1)..(1281)
atgaaagtattttttaaaataacaactttattgttaatgctaataagctatcaatcacttgctgcatttaataataatccatcgagtgtaggcgccgacagctcggggacataccgtaaccttgcacaagaaatgggtaaaacgaacatacagcaaaaggtgaatagtacttttaacaatatgtttggaagtaataatacccagcaattgtatacccattactcagaaaatgacgcatataaagcacactacataaaagcaattaaccctgacgagggtgatgatataagaacagaagggcaatcatgggggatgactgctgctgttatgctctacaagcaagaagaatttgataacttatggcgctttgctaaagcgtatcaaaaaaatccagacaatcaccctgatgtaaaaaaacaaggtgtttacgcgtggaaactaaagcttaatcaaaacggttttgtttataaagtagatgaaggcccggcacctgatggtgaagagtactttgcttttgcactacttaatgcgtcagctcgttggggtaattcgggcgaatttgattattatgctgatgccattaccatgttaaacacaattaaaaacaagctgatgcaaaaccaaataatccgtttttcaccttacattgataacctaacagacccttcttatcatatacctgcgttttacgactactttgcaaataacgtgactaatcaagccgataaaaattactggcgacaagtggctacaaaaagtagaaccttacttaaaaaccattttacaaaagtgaatgataatccgcattggaacttacctacatttttatcgcgcttagatggcagccctgttattggttacatttttaacggccaagcaaacccaggtcaatggtatgagtttgatgcatggcgcgttattatgaatgttggcttagacgcgcacttaatgggtgctcaggcgtggcataaaaatgcagttaataaagcactgggctttttaagttatgcaaaaacaaacaacagtaaaaactgctacgagcaagtgtattcatacggtggagcgcaaaacaaaggctgtgcaggcgaaggtcaaaaagccgcgaatgcagtagcgttacttgcttcaacaaacgctgggcaagcaaatgaattttttaacgagttttggtctttatcgcaaccaacaggtagctaccgttactacaatggttcgttatatatgttagctatgctgcatgtatcgggcaattttaagttttataacaacacgtttaattaa

SEQ ID NO: 4 - protein, 426 aa, Pseudoalteromonas sp.
MKVFFKITTLLLMLISYQSLAAFNNNPSSVGADSSGTYRNLAQEMGKTNIQQKVNSTFNNMFGSNNTQQLYTHYSENDAYKAHYIKAINPDEGDDIRTEGQSWGMTAAVMLYKQEEFDNLWRFAKAYQKNPDNHPDVKKQGVYAWKLKLNQNGFVYKVDEGPAPDGEEYFAFALLNASARWGNSGEFDYYADAITMLNTIKNKLMQNQIIRFSPYIDNLTDPSYHIPAFYDYFANNVTNQADKNYWRQVATKSRTLLKNHFTKVNDNPHWNLPTFLSRLDGSPVIGYIFNGQANPGQWYEFDAWRVIMNVGLDAHLMGAQAWHKNAVNKALGFLSYAKTNNSKNCYEQVYSYGGAQNKGCAGEGQKAANAVALLASTNAGQANEFFNEFWSLSQPTGSYRYYNGSLYMLAMLHVSGNFKFYNNTFN

SEQ ID NO: 5 - DNA, 1281 bp, Pseudoalteromonas sp.
  misc_feature: Xylanase sequence obtained from microbial strain TAH 4a
  CDS (1)..(1281)
atgaaagtattttttaaaataacaactttattgttaatgctaataagctatcaatcacttgctgcatttaataataatccatcgagtgtaggcgccgacagctcggggacataccgtaaccttgcacaagaaatgggtaaaacgaacatacagcaaaaggtgaatagtacttttaacaatatgtttggaagtaataatacccagcaattgtattacccatactcagaaaatgacgcatataaaacacactacataaaagcaattaaccctgacgagggtgatgatataagaacagaagggcaatcatgggggatgactgctgctgttatgctctacaagcaagaagaatttgataacttatggcgctttgctaaagcgtatcaaaaaaatccagacaatcaccctgatgtaaaaaaacaaggtgtttacgcgtggaaactaaagcttaatcaaaacggttttgtttataaagtagatgaaggcccggcacctgatggtgaagagtactttgcttttgcactacttaatgcgtcagctcgttggggtaattcgggcgaatttgattattatgctgatgccattaccatgttaaacacaattaaaaacaagctgatgcaaaaccaaataatccgtttttcaccttacattgataacctaacagacccttcttatcatatacctgcgttttacgactactttgcaaataacgtgactaatcaagccgataaaaattactggcgacaagtggctacaaaaagtagaaccttacttaaaaaccattttacaaaagtgaatgataatccgcattggaacttacctacatttttatcgcgcttagatggcagccctgttattggttacatttttaacggccaagcaaacccaggtcaatggtatgagtttgatgcatggcgcgttattatgaatgttggcttagacgcgcacttaatgggtgctcaggcgtggcataaaaatgcagttaataaagcacttggctttttaagttatgcaaaaacaaacaacagtaaaaactgttacgagcaagtgtattcgtacggtggagcgcaaaacaaaggctgcgcaggcgaaggtcaaaaagcagcaaatgcagtagcgttacttgcttcaacaaacactgggcaagcaaatgagttttttaacgacttttggtctttatcgcaaccaacgggtgactaccgttactacaatggttcgttatatatgttagctatgctgcatgtatcgggtaactttaagttttacaacaacacgtttaattaa

SEQ ID NO: 6 - protein, 426 aa, Pseudoalteromonas sp.
MKVFFKITTLLLMLISYQSLAAFNNNPSSVGADSSGTYRNLAQEMGKTNIQQKVNSTFNNMFGSNNTQQLYYPYSENDAYKTHYIKAINPDEGDDIRTEGQSWGMTAAVMLYKQEEFDNLWRFAKAYQKNPDNHPDVKKQGVYAWKLKLNQNGFVYKVDEGPAPDGEEYFAFALLNASARWGNSGEFDYYADAITMLNTIKNKLMQNQIIRFSPYIDNLTDPSYHIPAFYDYFANNVTNQADKNYWRQVATKSRTLLKNHFTKVNDNPHWNLPTFLSRLDGSPVIGYIFNGQANPGQWYEFDAWRVIMNVGLDAHLMGAQAWHKNAVNKALGFLSYAKTNNSKNCYEQVYSYGGAQNKGCAGEGQKAANAVALLASTNTGQANEFFNDFWSLSQPTGDYRYYNGSLYMLAMLHVSGNFKFYNNTFN

SEQ ID NO: 7 - DNA, 1281 bp, Pseudoalteromonas sp.
  misc_feature: Xylanase sequence obtained from microbial strain TAH 7a
  CDS (1)..(1281)
atgaaagtattttttaaaataacaactttattgttaatgctaataagctatcaatcacttgctgcatttaataataatcctacgagtgtaggccccgcaagcttggggacataccgttaccttgcacaagaattgcgtaaacggaccatacgccaaagcgtgaatagtacttttaacaatatgtttggaagtaataatacccttcacccgtattacccatactcagaaaatgacgcatataaagcacactacataaaagcaattaaccctgacgagggtgatgatataagaacagaagggcaatcatgggggatgactgctgctgttatgctctacaagcaagaagaatttgataacttatggcgctttgctaaagcgtatcaaaaaaatccagccaatcaccctgatgtaaaaaaacaaggtgtttacgcgtggaaactaaagcttaatcaaaacggttttgtttataaagtagatgaaggcccggcacctgatggtgaagagtactttgcttttgcactacttaatgcgtcagctcgttggggtaattcgggcgaatttgattattatgctgatgccattaccatgttaaacacaattaaaaacaagctgatgcaaaaccaaataatccgtttttcaccttacattgataacctaacagacccttcttatcatatacctgcgttttacgactactttgcaaataacgtgactaatcaagccgataaaaattactggcgacaagtggctacaaaaagtagaaccttacttaaaaaccattttacaaaagtgaatgataatccgcattggaacttacctacatttttatcgcgcttagatggcagccctgttattggttacatttttaacggccaagcaaacccaggtcaatggtatgagtttgatgcatggcgcgttattatgaatgttggcttagacgcgcacttaatgggtgctcaggcgtggcataaaaatgcagttaataaagcacttggctttttaagttatgcaaaaacaaacaacagtaaaaactgttacgagcaagtgtattcgtacggtggagcgcaaaacaaaggctgcgcaggcgaaggtcaaaaagcagcaaatgcagtagcgttacttgcttcaacaaacactgggcaagccaatgagttttttaacgactttgggtctttatcgcaaccaacgggtgactaccgttactacaatggttcgttatatatgttagctatgctgcatgtatcggggaactttaagttttacaacaacacgtttaattaa

SEQ ID NO: 8 - protein, 426 aa, Pseudoalteromonas sp.
MKVFFKITTLLLMLISYQSLAAFNNNPTSVGPASLGTYRYLAQELRKRTIRQSVNSTFNNMFGSNNTLHPYYPYSENDAYKAHYIKAINPDEGDDIRTEGQSWGMTAAVMLYKQEEFDNLWRFAKAYQKNPANHPDVKKQGVYAWKLKLNQNGFVYKVDEGPAPDGEEYFAFALLNASARWGNSGEFDYYADAITMLNTIKNKLMQNQIIRFSPYIDNLTDPSYHIPAFYDYFANNVTNQADKNYWRQVATKSRTLLKNHFTKVNDNPHWNLPTFLSRLDGSPVIGYIFNGQANPGQWYEFDAWRVIMNVGLDAHLMGAQAWHKNAVNKALGFLSYAKTNNSKNCYEQVYSYGGAQNKGCAGEGQKAANAVALLASTNTGQANEFFNDFGSLSQPTGDYRYYNGSLYMLAMLHVSGNFKFYNNTFN

SEQ ID NO: 9 - DNA, 1281 bp, Unknown
  Xylanase sequence obtained from microbial strain Sp.10.1
  CDS (1)..(1281)
atgaaagtattttttaaaataacaactttattgttaatactaataagctatcaatcacttgctgcatttaataataacccatcgagtgtaggcgccgacagttcaggagcataccgtaacctcgcacaagaaatgggtaaaacaaatatacagcaaaaagtgaatagtacttttgacaatatgtttggctataacaacacacaacaactttactacccgtacaccgaaaacggtgtttataaagcacactacataaaagcaattaaccctgacgagggtgatgatataagaacagaagggcaatcgtggggaatgaccgccgctgtcatgcttaataaacaagaagaatttgataacctatggcgctttgcaaaagcgtatcaaaaaaatccagacaaccaccctgatgctaaaaaacaaggcgtttacgcgtggaaactaaagcttaatcaaaacggctttgtttataaagtagatgaaggccccgctcctgatggcgaagagtactttgcttttgcactacttaatgcgtcagctcgttggggtaattcgggggagtttaactactacaacgatgctattatcatgttaaatacaattaaaaataagctgatggataaccaaataatccgcttttcgccttacattgataacttaacagacccttcttaccatatacctgcgttttacgactactttgctaataacgtaactaaccaagcagatcaaacttactggcgacaagtagccacaaaaagtagaaccttacttaaaaaccattttacgaaagtaagtgataatccgcattggaatttacctacatttttatcgcgcttagatggcagccctgttattggctacatttttaacggccaagcaaacccaggtcaatggtatgaatttgatgcatggcgcgtaattatgaatgtgggtttagacgcacatttaatgggtgctcaagcatggcataaaagtgcagtaaataaagcattaggctttttaagttatgcaaaaacaaacaacagtaaatactgttacgagcaagtgtattcgtacggtggagcgcaaaacaaaggctgtgcaggcgaaggtcaaaaagccgcgaatgcagtagcgttacttgcttcaacaaacgctgggcaagcaaatgaattttttaacgagttttggtctttatcgcaaccaacaggtagctaccgttactacaatggttcgttatatatgttagctatgctgcatgtatcgggcaattttaagttttataacaacacgtttaattaa

SEQ ID NO: 10 - protein, 426 aa, Artificial sequence
  Synthetic Construct
MKVFFKITTLLLILISYQSLAAFNNNPSSVGADSSGAYRNLAQEMGKTNIQQKVNSTFDNMFGYNNTQQLYYPYTENGVYKAHYIKAINPDEGDDIRTEGQSWGMTAAVMLNKQEEFDNLWRFAKAYQKNPDNHPDAKKQGVYAWKLKLNQNGFVYKVDEGPAPDGEEYFAFALLNASARWGNSGEFNYYNDAIIMLNTIKNKLMDNQIIRFSPYIDNLTDPSYHIPAFYDYFANNVTNQADQTYWRQVATKSRTLLKNHFTKVSDNPHWNLPTFLSRLDGSPVIGYIFNGQANPGQWYEFDAWRVIMNVGLDAHLMGAQAWHKSAVNKALGFLSYAKTNNSKYCYEQVYSYGGAQNKGCAGEGQKAANAVALLASTNAGQANEFFNEFWSLSQPTGSYRYYNGSLYMLAMLHVSGNFKFYNNTFN

SEQ ID NO: 11 - DNA, 1281 bp, Unknown
  Xylanase sequence obtained from microbial strain Sp.10.2
  CDS (1)..(1281)
atgaaagtattttttaaaataacaactttattgttaacactaataagctatcaatcacttgctgcatttaatagtaacccatcgagtgtaggcgccgacagttcaggagcataccgtaacctcgcacaagaaatgggtaaaacaaatatacagcaaaaagtgaatagtacttttgacaatatgtttggctataacaacacacaacaactttactacccgtacaccgaaaacggtgtttataaagcacactacataaaagcaattaacccagacgaaggcgacgatataagaacagaagggcaatcgtggggaatgaccgctgctgtcatgcttaataaacaagaagaatttgataacctatggcgctttgcaaaagcgtatcaaaaaaatccagacaaccaccctgatgctaaaaaacaaggcgtttacgcgtggaaactaaagcttaatcaaaacggctttgtttataaagtagatgaaggccccgctccggatggcgaagagtactttgcttttgcactacttaatgcgtcagctcgttggggtaattcgggggagtttaactactacaacgatgctattatcatgttaaatacaattaaaaataagctgatggaaaaccaaataatccgcttttcgccttacattgataacttaacagacccttcttaccatatacctgcgttttacgactactttgctaataacgtaactaaccaagcagacaaaaattactggcgacaagtagccacaaaaagtagaaccttacttaaaaaccattttacgaaagtaagtgataatccgcattggaatttacctacatttttatcgcgcttagatggcagccctgttattggctacatttttaacggccaagcaaacccaggtcaatggtatgaatttgatgcatggcgcgtaattatgaatgtgggtttagacgcgcatttaatgggtgctcaagcatggcataaaagtgcagtaaataaagcattaggctttttaagttatgcaaaaacaaacaacagtaaaaactgttacgagcaagtgtattcgtacggtggagcgcaaaacaaaggttgtgcaggcgaaggtcaaaaagccgcgaatgcagtagcgttacttgcttcaacaaatgctgggcaagcaaatgaattttttaacgagttttggtctttatcgcaaccaacaggtaactaccgttactacaatggttcgctgtatatgttagctatgctgcatgtctcgggcaattttaagttttataacaacacgtttaattaa

SEQ ID NO: 12 - protein, 426 aa, Artificial sequence
  Synthetic Construct
MKVFFKITTLLLTLISYQSLAAFNSNPSSVGADSSGAYRNLAQEMGKTNIQQKVNSTFDNMFGYNNTQQLYYPYTENGVYKAHYIKAINPDEGDDIRTEGQSWGMTAAVMLNKQEEFDNLWRFAKAYQKNPDNHPDAKKQGVYAWKLKLNQNGFVYKVDEGPAPDGEEYFAFALLNASARWGNSGEFNYYNDAIIMLNTIKNKLMENQIIRFSPYIDNLTDPSYHIPAFYDYFANNVTNQADKNYWRQVATKSRTLLKNHFTKVSDNPHWNLPTFLSRLDGSPVIGYIFNGQANPGQWYEFDAWRVIMNVGLDAHLMGAQAWHKSAVNKALGFLSYAKTNNSKNCYEQVYSYGGAQNKGCAGEGQKAANAVALLASTNAGQANEFFNEFWSLSQPTGNYRYYNGSLYMLAMLHVSGNFKFYNNTFN

SEQ ID NO: 13 - DNA, 1281 bp, Unknown
  Xylanase sequence obtained from microbial strain Sp.11.2
  CDS (1)..(1281)
atgaaagtattttttaaaataacaactttattgttaacgctaataagctatcaatcacttgctgcatttaataataacccatcgagtgtaggcgccgacagttcagggacataccgtaacctcgcacaagaaatgggtaaaacaaatatacagcaaaaggtgaatagtacttttgacaatatgtttggctataacaacactcaacaactttactacccgtacaccgaaaacggtgtttataaagcacactacataaaagcaattaacccagacgaaggcgacgatataagaacagaagggcaatcgtggggaatgaccgccgctgtcatgcttaacaaacaagaagaatttgataacctatggcgctttgcaaaagcgtatcaaaaaaatccagacaatcaccctgatgctaaaaaacaaggcgtttacgcgtggaaactaaagcttaatcaaaacggctttgtttataaagtggatgagggccccgctcccgatggtgaagagtactttgcgtttgcactacttaatgcctctgctcgttgggggaattcgggtgagtttaactactacaacgatgccattaccatgttaaacacaattaaaaataagctgatggaaaaccaaataatccgcttttcaccttatttagataacttaacagatccttcataccatatacctgcgttttatgactactttgcaaataacgtaactaaccaagcagacaaaacttactggcgacaagtagccacaaaaagtagaaccttacttaaaaaccattttacaaaagtaagtggtagcccgcattggaacttacctacatttttatcgcgcttagatggcagccctgttattggctacatttttaacgaccaagcaaacccaggtcaatggtatgaatttgatgcatggcgcgtaattatgaatgtgggtttagacgcgcatttaatgggtgctcaagcgtggcataaaagcgcagtaaataaagcactgggctttttaagttatgcaaaaacaaacaacagtaaaaactgctacgagcaagtgtattcatacggtggagcgcaaaacaaaggctgtgcaggcgaaggtcaaaaagccgcgaatgcagtagcgttacttgcttcaacaaacgctgggcaagcaaatgaattttttaacgagttttggtctttatcgcaaccaacaggtagctaccgttactacaatggttcgttatatatgttagctatgctgcatgtatcgggcaattttaagttttataacaacacgtttaattaa

SEQ ID NO: 14 - protein, 426 aa, Artificial sequence
  Synthetic Construct
MKVFFKITTLLLTLISYQSLAAFNNNPSSVGADSSGTYRNLAQEMGKTNIQQKVNSTFDNMFGYNNTQQLYYPYTENGVYKAHYIKAINPDEGDDIRTEGQSWGMTAAVMLNKQEEFDNLWRFAKAYQKNPDNHPDAKKQGVYAWKLKLNQNGFVYKVDEGPAPDGEEYFAFALLNASARWGNSGEFNYYNDAITMLNTIKNKLMENQIIRFSPYLDNLTDPSYHIPAFYDYFANNVTNQADKTYWRQVATKSRTLLKNHFTKVSGSPHWNLPTFLSRLDGSPVIGYIFNDQANPGQWYEFDAWRVIMNVGLDAHLMGAQAWHKSAVNKALGFLSYAKTNNSKNCYEQVYSYGGAQNKGCAGEGQKAANAVALLASTNAGQANEFFNEFWSLSQPTGSYRYYNGSLYMLAMLHVSGNFKFYNNTFN

SEQ ID NO: 15 - DNA, 1281 bp, Unknown
  Xylanase sequence obtained from microbial strain Sp.23.2
  CDS (1)..(1281)
atgaaagtattttttaaaataacaactttattgttaatactaataagctatcaatcacttgctgcatttaataataacccatcgagtgtaggcgcctacagttcagggacataccgtaacctcgcacaagaaatgggtaaaacaaatatacagcaaaaagtgaatagtacttttgacaatatgtttggctataacaacacacaacaactttactacccgtacaccgaaaacggtgtttataaagcacactacataaaagcaattaacccagacgaaggcgacgatataagaacagaagggcaatcgtggggaatgactgccgctatcatgcttaataaacaagaagaatttgataatctatggcgctttgcaaaagcgtatcaaaaaaatccagacaaccaccctgatgtaaaaaaacaaggcgtttacgcgtggaaactaaagcttaatcaaaacggctttgtttataaagtagatgaaggccccgctccggatggcgaagagtactttgcgtttgcactacttaatgcctccgctcgttggggtaattcaggggagtttaactactacaacgatgcaattaccatgttaaacacaattaaaaataagctgatggaaaaccaaataatacgcttttcgccttacattgataacctaacagacccttcttaccatatacctgcgttttacgactactttgctaataacgtaactaaccaagcagacaaaacttactggcgacaagtagccacaaaaagtagaaccttactcaaaaaccattttacaaaagtaagtggtagcccgcattggaacttacctacatttttatcgcgcttagatggcagccctgttattggctacatttttaacggccaagcaaacccaggtcaatggtatgaatttgatgcatgccgcgtatttatgaatgtggggttagaggcgcattttatgggtgctcaagcgtggcataaaagtgcagttaataaagcactgggctttttaagttatgcaaaaacaaacaacagtaaaaactgttacgagcaagtgtattcgtacggtggagcgcaaaacagaggctgtgcaggcgaaggtcaaaaagccgcgaatgcagtagcgttacttgcttcaacaaatgctgggcaagcaaatgagttttttaacgaattttggtctttatcgcaaccaacgggtgactaccgttactataatggttcgttatatatgttagctatgctgcatgtatcgggcaattttaagttttataacaacacgtttaattaa

SEQ ID NO: 16 - protein, 426 aa, Artificial sequence
  Synthetic Construct
MKVFFKITTLLLILISYQSLAAFNNNPSSVGAYSSGTYRNLAQEMGKTNIQQKVNSTFDNMFGYNNTQQLYYPYTENGVYKAHYIKAINPDEGDDIRTEGQSWGMTAAIMLNKQEEFDNLWRFAKAYQKNPDNHPDVKKQGVYAWKLKLNQNGFVYKVDEGPAPDGEEYFAFALLNASARWGNSGEFNYYNDAITMLNTIKNKLMENQIIRFSPYIDNLTDPSYHIPAFYDYFANNVTNQADKTYWRQVATKSRTLLKNHFTKVSGSPHWNLPTFLSRLDGSPVIGYIFNGQANPGQWYEFDACRVFMNVGLEAHFMGAQAWHKSAVNKALGFLSYAKTNNSKNCYEQVYSYGGAQNRGCAGEGQKAANAVALLASTNAGQANEFFNEFWSLSQPTGDYRYYNGSLYMLAMLHVSGNFKFYNNTFN

SEQ ID NO: 17 - DNA, 34 bp, Artificial Sequence
  Sense primer
gggcatatgaaagtattttttaaaataacaactt

SEQ ID NO: 18 - DNA, 33 bp, Artificial Sequence
  Antisense primer
gggctcgagttaattaaacgtgttgttataaaa

SEQ ID NO: 19 - DNA, 162 bp, Artificial Sequence
  Consensus sequence for microbial strains TAH 2a, TAH 4a, TAH 7a, Sp.10.1, Sp.10.2 and Sp.23.2
  misc_feature (24)..(24): n is a, c, g, or t
  misc_feature (27)..(27): n is a, c, g, or t
  misc_feature (48)..(48): n is a, c, g, or t
  misc_feature (51)..(51): n is a, c, g, or t
  misc_feature (81)..(82): n is a, c, g, or t
  misc_feature (93)..(93): n is a, c, g, or t
  misc_feature (99)..(99): n is a, c, g, or t
  misc_feature (111)..(111): n is a, c, g, or t
  misc_feature (115)..(115): n is a, c, g, or t
  misc_feature (135)..(135): n is a, c, g, or t
  misc_feature (139)..(140): n is a, c, g, or t
tacggtggagcgcaaaacaaaggntgngcaggcgaaggtcaaaaagcngcnaatgcagtagcgttacttgcttcaacaaannctgggcaagcnaatganttttttaacgantttnggtctttatcgcaaccaacnggtnnctaccgttactacaatggttcg

SEQ ID NO: 20 - DNA, 1218 bp, Pseudoalteromonas sp.
  misc_feature: Xylanase sequence (mature protein) obtained from microbial strain TAH 2a
  CDS (1)..(1218)
gcatttaataataatccatcgagtgtaggcgccgacagctcggggacataccgtaaccttgcacaagaaatgggtaaaacgaacatacagcaaaaggtgaatagtacttttaacaatatgtttggaagtaataatacccagcaattgtatacccattactcagaaaatgacgcatataaagcacactacataaaagcaattaaccctgacgagggtgatgatataagaacagaagggcaatcatgggggatgactgctgctgttatgctctacaagcaagaagaatttgataacttatggcgctttgctaaagcgtatcaaaaaaatccagacaatcaccctgatgtaaaaaaacaaggtgtttacgcgtggaaactaaagcttaatcaaaacggttttgtttataaagtagatgaaggcccggcacctgatggtgaagagtactttgcttttgcactacttaatgcgtcagctcgttggggtaattcgggcgaatttgattattatgctgatgccattaccatgttaaacacaattaaaaacaagctgatgcaaaaccaaataatccgtttttcaccttacattgataacctaacagacccttcttatcatatacctgcgttttacgactactttgcaaataacgtgactaatcaagccgataaaaattactggcgacaagtggctacaaaaagtagaaccttacttaaaaaccattttacaaaagtgaatgataatccgcattggaacttacctacatttttatcgcgcttagatggcagccctgttattggttacatttttaacggccaagcaaacccaggtcaatggtatgagtttgatgcatggcgcgttattatgaatgttggcttagacgcgcacttaatgggtgctcaggcgtggcataaaaatgcagttaataaagcactgggctttttaagttatgcaaaaacaaacaacagtaaaaactgctacgagcaagtgtattcatacggtggagcgcaaaacaaaggctgtgcaggcgaaggtcaaaaagccgcgaatgcagtagcgttacttgcttcaacaaacgctgggcaagcaaatgaattttttaacgagttttggtctttatcgcaaccaacaggtagctaccgttactacaatggttcgttatatatgttagctatgctgcatgtatcgggcaattttaagttttataacaacacgtttaattaa

SEQ ID NO: 21 - protein, 405 aa, Pseudoalteromonas sp.
AFNNNPSSVGADSSGTYRNLAQEMGKTNIQQKVNSTFNNMFGSNNTQQLYTHYSENDAYKAHYIKAINPDEGDDIRTEGQSWGMTAAVMLYKQEEFDNLWRFAKAYQKNPDNHPDVKKQGVYAWKLKLNQNGFVYKVDEGPAPDGEEYFAFALLNASARWGNSGEFDYYADAITMLNTIKNKLMQNQIIRFSPYIDNLTDPSYHIPAFYDYFANNVTNQADKNYWRQVATKSRTLLKNHFTKVNDNPHWNLPTFLSRLDGSPVIGYIFNGQANPGQWYEFDAWRVIMNVGLDAHLMGAQAWHKNAVNKALGFLSYAKTNNSKNCYEQVYSYGGAQNKGCAGEGQKAANAVALLASTNAGQANEFFNEFWSLSQPTGSYRYYNGSLYMLAMLHVSGNFKFYNNTFN

SEQ ID NO: 22 - DNA, 1218 bp, Pseudoalteromonas sp.
  misc_feature: Xylanase sequence (mature protein) obtained from microbial strain TAH 4a
  CDS (1)..(1218)
gcatttaataataatccatcgagtgtaggcgccgacagctcggggacataccgtaaccttgcacaagaaatgggtaaaacgaacatacagcaaaaggtgaatagtacttttaacaatatgtttggaagtaataatacccagcaattgtattacccatactcagaaaatgacgcatataaaacacactacataaaagcaattaaccctgacgagggtgatgatataagaacagaagggcaatcatgggggatgactgctgctgttatgctctacaagcaagaagaatttgataacttatggcgctttgctaaagcgtatcaaaaaaatccagacaatcaccctgatgtaaaaaaacaaggtgtttacgcgtggaaactaaagcttaatcaaaacggttttgtttataaagtagatgaaggcccggcacctgatggtgaagagtactttgcttttgcactacttaatgcgtcagctcgttggggtaattcgggcgaatttgattattatgctgatgccattaccatgttaaacacaattaaaaacaagctgatgcaaaaccaaataatccgtttttcaccttacattgataacctaacagacccttcttatcatatacctgcgttttacgactactttgcaaataacgtgactaatcaagccgataaaaattactggcgacaagtggctacaaaaagtagaaccttacttaaaaaccattttacaaaagtgaatgataatccgcattggaacttacctacatttttatcgcgcttagatggcagccctgttattggttacatttttaacggccaagcaaacccaggtcaatggtatgagtttgatgcatggcgcgttattatgaatgttggcttagacgcgcacttaatgggtgctcaggcgtggcataaaaatgcagttaataaagcacttggctttttaagttatgcaaaaacaaacaacagtaaaaactgttacgagcaagtgtattcgtacggtggagcgcaaaacaaaggctgcgcaggcgaaggtcaaaaagcagcaaatgcagtagcgttacttgcttcaacaaacactgggcaagcaaatgagttttttaacgacttttggtctttatcgcaaccaacgggtgactaccgttactacaatggttcgttatatatgttagctatgctgcatgtatcgggtaactttaagttttacaacaacacgtttaattaa

SEQ ID NO: 23 - protein, 405 aa, Pseudoalteromonas sp.
AFNNNPSSVGADSSGTYRNLAQEMGKTNIQQKVNSTFNNMFGSNNTQQLYYPYSENDAYKTHYIKAINPDEGDDIRTEGQSWGMTAAVMLYKQEEFDNLWRFAKAYQKNPDNHPDVKKQGVYAWKLKLNQNGFVYKVDEGPAPDGEEYFAFALLNASARWGNSGEFDYYADAITMLNTIKNKLMQNQIIRFSPYIDNLTDPSYHIPAFYDYFANNVTNQADKNYWRQVATKSRTLLKNHFTKVNDNPHWNLPTFLSRLDGSPVIGYIFNGQANPGQWYEFDAWRVIMNVGLDAHLMGAQAWHKNAVNKALGFLSYAKTNNSKNCYEQVYSYGGAQNKGCAGEGQKAANAVALLASTNTGQANEFFNDFWSLSQPTGDYRYYNGSLYMLAMLHVSGNFKFYNNTFN

SEQ ID NO: 24 - DNA, 1218 bp, Pseudoalteromonas sp.
  misc_feature: Xylanase sequence (mature protein) obtained from microbial strain TAH 7a
  CDS (1)..(1218)
gcatttaataataatcctacgagtgtaggccccgcaagcttggggacataccgttaccttgcacaagaattgcgtaaacggaccatacgccaaagcgtgaatagtacttttaacaatatgtttggaagtaataatacccttcacccgtattacccatactcagaaaatgacgcatataaagcacactacataaaagcaattaaccctgacgagggtgatgatataagaacagaagggcaatcatgggggatgactgctgctgttatgctctacaagcaagaagaatttgataacttatggcgctttgctaaagcgtatcaaaaaaatccagccaatcaccctgatgtaaaaaaacaaggtgtttacgcgtggaaactaaagcttaatcaaaacggttttgtttataaagtagatgaaggcccggcacctgatggtgaagagtactttgcttttgcactacttaatgcgtcagctcgttggggtaattcgggcgaatttgattattatgctgatgccattaccatgttaaacacaattaaaaacaagctgatgcaaaaccaaataatccgtttttcaccttacattgataacctaacagacccttcttatcatatacctgcgttttacgactactttgcaaataacgtgactaatcaagccgataaaaattactggcgacaagtggctacaaaaagtagaaccttacttaaaaaccattttacaaaagtgaatgataatccgcattggaacttacctacatttttatcgcgcttagatggcagccctgttattggttacatttttaacggccaagcaaacccaggtcaatggtatgagtttgatgcatggcgcgttattatgaatgttggcttagacgcgcacttaatgggtgctcaggcgtggcataaaaatgcagttaataaagcacttggctttttaagttatgcaaaaacaaacaacagtaaaaactgttacgagcaagtgtattcgtacggtggagcgcaaaacaaaggctgcgcaggcgaaggtcaaaaagcagcaaatgcagtagcgttacttgcttcaacaaacactgggcaagccaatgagttttttaacgactttgggtctttatcgcaaccaacgggtgactaccgttactacaatggttcgttatatatgttagctatgctgcatgtatcggggaactttaagttttacaacaacacgtttaattaa

SEQ ID NO: 25 - protein, 405 aa, Pseudoalteromonas sp.
AFNNNPTSVGPASLGTYRYLAQELRKRTIRQSVNSTFNNMFGSNNTLHPYYPYSENDAYKAHYIKAINPDEGDDIRTEGQSWGMTAAVMLYKQEEFDNLWRFAKAYQKNPANHPDVKKQGVYAWKLKLNQNGFVYKVDEGPAPDGEEYFAFALLNASARWGNSGEFDYYADAITMLNTIKNKLMQNQIIRFSPYIDNLTDPSYHIPAFYDYFANNVTNQADKNYWRQVATKSRTLLKNHFTKVNDNPHWNLPTFLSRLDGSPVIGYIFNGQANPGQWYEFDAWRVIMNVGLDAHLMGAQAWHKNAVNKALGFLSYAKTNNSKNCYEQVYSYGGAQNKGCAGEGQKAANAVALLASTNTGQANEFFNDFGSLSQPTGDYRYYNGSLYMLAMLHVSGNFKFYNNTFN

SEQ ID NO: 26 - DNA, 1218 bp, Unknown
  Xylanase sequence (mature protein) obtained from microbial strain Sp.10.1
  CDS (1)..(1218)
gcatttaataataacccatcgagtgtaggcgccgacagttcaggagcataccgtaacctcgcacaagaaatgggtaaaacaaatatacagcaaaaagtgaatagtacttttgacaatatgtttggctataacaacacacaacaactttactacccgtacaccgaaaacggtgtttataaagcacactacataaaagcaattaaccctgacgagggtgatgatataagaacagaagggcaatcgtggggaatgaccgccgctgtcatgcttaataaacaagaagaatttgataacctatggcgctttgcaaaagcgtatcaaaaaaatccagacaaccaccctgatgctaaaaaacaaggcgtttacgcgtggaaactaaagcttaatcaaaacggctttgtttataaagtagatgaaggccccgctcctgatggcgaagagtactttgcttttgcactacttaatgcgtcagctcgttggggtaattcgggggagtttaactactacaacgatgctattatcatgttaaatacaattaaaaataagctgatggataaccaaataatccgcttttcgccttacattgataacttaacagacccttcttaccatatacctgcgttttacgactactttgctaataacgtaactaaccaagcagatcaaacttactggcgacaagtagccacaaaaagtagaaccttacttaaaaaccattttacgaaagtaagtgataatccgcattggaatttacctacatttttatcgcgcttagatggcagccctgttattggctacatttttaacggccaagcaaacccaggtcaatggtatgaatttgatgcatggcgcgtaattatgaatgtgggtttagacgcacatttaatgggtgctcaagcatggcataaaagtgcagtaaataaagcattaggctttttaagttatgcaaaaacaaacaacagtaaatactgttacgagcaagtgtattcgtacggtggagcgcaaaacaaaggctgtgcaggcgaaggtcaaaaagccgcgaatgcagtagcgttacttgcttcaacaaacgctgggcaagcaaatgaattttttaacgagttttggtctttatcgcaaccaacaggtagctaccgttactacaatggttcgttatatatgttagctatgctgcatgtatcgggcaattttaagttttataacaacacgtttaattaa

SEQ ID NO: 27 - protein, 405 aa, Artificial sequence
  Synthetic Construct
AFNNNPSSVGADSSGAYRNLAQEMGKTNIQQKVNSTFDNMFGYNNTQQLYYPYTENGVYKAHYIKAINPDEGDDIRTEGQSWGMTAAVMLNKQEEFDNLWRFAKAYQKNPDNHPDAKKQGVYAWKLKLNQNGFVYKVDEGPAPDGEEYFAFALLNASARWGNSGEFNYYNDAIIMLNTIKNKLMDNQIIRFSPYIDNLTDPSYHIPAFYDYFANNVTNQADQTYWRQVATKSRTLLKNHFTKVSDNPHWNLPTFLSRLDGSPVIGYIFNGQANPGQWYEFDAWRVIMNVGLDAHLMGAQAWHKSAVNKALGFLSYAKTNNSKYCYEQVYSYGGAQNKGCAGEGQKAANAVALLASTNAGQANEFFNEFWSLSQPTGSYRYYNGSLYMLAMLHVSGNFKFYNNTFN

SEQ ID NO: 28 - DNA, 1218 bp, Unknown
  Xylanase sequence (mature protein) obtained from microbial strain Sp.10.2
  CDS (1)..(1218)
gcatttaatagtaacccatcgagtgtaggcgccgacagttcaggagcataccgtaacctcgcacaagaaatgggtaaaacaaatatacagcaaaaagtgaatagtacttttgacaatatgtttggctataacaacacacaacaactttactacccgtacaccgaaaacggtgtttataaagcacactacataaaagcaattaacccagacgaaggcgacgatataagaacagaagggcaatcgtggggaatgaccgctgctgtcatgcttaataaacaagaagaatttgataacctatggcgctttgcaaaagcgtatcaaaaaaatccagacaaccaccctgatgctaaaaaacaaggcgtttacgcgtggaaactaaagcttaatcaaaacggctttgtttataaagtagatgaaggccccgctccggatggcgaagagtactttgcttttgcactacttaatgcgtcagctcgttggggtaattcgggggagtttaactactacaacgatgctattatcatgttaaatacaattaaaaataagctgatggaaaaccaaataatccgcttttcgccttacattgataacttaacagacccttcttaccatatacctgcgttttacgactactttgctaataacgtaactaaccaagcagacaaaaattactggcgacaagtagccacaaaaagtagaaccttacttaaaaaccattttacgaaagtaagtgataatccgcattggaatttacctacatttttatcgcgcttagatggcagccctgttattggctacatttttaacggccaagcaaacccaggtcaatggtatgaatttgatgcatggcgcgtaattatgaatgtgggtttagacgcgcatttaatgggtgctcaagcatggcataaaagtgcagtaaataaagcattaggctttttaagttatgcaaaaacaaacaacagtaaaaactgttacgagcaagtgtattcgtacggtggagcgcaaaacaaaggttgtgcaggcgaaggtcaaaaagccgcgaatgcagtagcgttacttgcttcaacaaatgctgggcaagcaaatgaattttttaacgagttttggtctttatcgcaaccaacaggtaactaccgttactacaatggttcgctgtatatgttagctatgctgcatgtctcgggcaattttaagttttataacaacacgtttaattaa

SEQ ID NO: 29 - protein, 405 aa, Artificial sequence
  Synthetic Construct
AFNSNPSSVGADSSGAYRNLAQEMGKTNIQQKVNSTFDNMFGYNNTQQLYYPYTENGVYKAHYIKAINPDEGDDIRTEGQSWGMTAAVMLNKQEEFDNLWRFAKAYQKNPDNHPDAKKQGVYAWKLKLNQNGFVYKVDEGPAPDGEEYFAFALLNASARWGNSGEFNYYNDAIIMLNTIKNKLMENQIIRFSPYIDNLTDPSYHIPAFYDYFANNVTNQADKNYWRQVATKSRTLLKNHFTKVSDNPHWNLPTFLSRLDGSPVIGYIFNGQANPGQWYEFDAWRVIMNVGLDAHLMGAQAWHKSAVNKALGFLSYAKTNNSKNCYEQVYSYGGAQNKGCAGEGQKAANAVALLASTNAGQANEFFNEFWSLSQPTGNYRYYNGSLYMLAMLHVSGNFKFYNNTFN

SEQ ID NO: 30 - DNA, 1218 bp, Unknown
  Xylanase sequence (mature protein) obtained from microbial strain Sp.11.2
  CDS (1)..(1218)
gcatttaataataacccatcgagtgtaggcgccgacagttcagggacataccgtaacctcgcacaagaaatgggtaaaacaaatatacagcaaaaggtgaatagtacttttgacaatatgtttggctataacaacactcaacaactttactacccgtacaccgaaaacggtgtttataaagcacactacataaaagcaattaacccagacgaaggcgacgatataagaacagaagggcaatcgtggggaatgaccgccgctgtcatgcttaacaaacaagaagaatttgataacctatggcgctttgcaaaagcgtatcaaaaaaatccagacaatcaccctgatgctaaaaaacaaggcgtttacgcgtggaaactaaagcttaatcaaaacggctttgtttataaagtggatgagggccccgctcccgatggtgaagagtactttgcgtttgcactacttaatgcctctgctcgttgggggaattcgggtgagtttaactactacaacgatgccattaccatgttaaacacaattaaaaataagctgatggaaaaccaaataatccgcttttcaccttatttagataacttaacagatccttcataccatatacctgcgttttatgactactttgcaaataacgtaactaaccaagcagacaaaacttactggcgacaagtagccacaaaaagtagaaccttacttaaaaaccattttacaaaagtaagtggtagcccgcattggaacttacctacatttttatcgcgcttagatggcagccctgttattggctacatttttaacgaccaagcaaacccaggtcaatggtatgaatttgatgcatggcgcgtaattatgaatgtgggtttagacgcgcatttaatgggtgctcaagcgtggcataaaagcgcagtaaataaagcactgggctttttaagttatgcaaaaacaaacaacagtaaaaactgctacgagcaagtgtattcatacggtggagcgcaaaacaaaggctgtgcaggcgaaggtcaaaaagccgcgaatgcagtagcgttacttgcttcaacaaacgctgggcaagcaaatgaattttttaacgagttttggtctttatcgcaaccaacaggtagctaccgttactacaatggttcgttatatatgttagctatgctgcatgtatcgggcaattttaagttttataacaacacgtttaattaa

SEQ ID NO: 31 - protein, 405 aa, Artificial sequence
  Synthetic Construct
AFNNNPSSVGADSSGTYRNLAQEMGKTNIQQKVNSTFDNMFGYNNTQQLYYPYTENGVYKAHYIKAINPDEGDDIRTEGQSWGMTAAVMLNKQEEFDNLWRFAKAYQKNPDNHPDAKKQGVYAWKLKLNQNGFVYKVDEGPAPDGEEYFAFALLNASARWGNSGEFNYYNDAITMLNTIKNKLMENQIIRFSPYLDNLTDPSYHIPAFYDYFANNVTNQADKTYWRQVATKSRTLLKNHFTKVSGSPHWNLPTFLSRLDGSPVIGYIFNDQANPGQWYEFDAWRVIMNVGLDAHLMGAQAWHKSAVNKALGFLSYAKTNNSKNCYEQVYSYGGAQNKGCAGEGQKAANAVALLASTNAGQANEFFNEFWSLSQPTGSYRYYNGSLYMLAMLHVSGNFKFYNNTFN

SEQ ID NO: 32 - DNA, 1218 bp, Unknown
  Xylanase sequence (mature protein) obtained from microbial strain Sp.23.2
  CDS (1)..(1218)
gcatttaataataacccatcgagtgtaggcgcctacagttcagggacataccgtaacctcgcacaagaaatgggtaaaacaaatatacagcaaaaagtgaatagtacttttgacaatatgtttggctataacaacacacaacaactttactacccgtacaccgaaaacggtgtttataaagcacactacataaaagcaattaacccagacgaaggcgacgatataagaacagaagggcaatcgtggggaatgactgccgctatcatgcttaataaacaagaagaatttgataatctatggcgctttgcaaaagcgtatcaaaaaaatccagacaaccaccctgatgtaaaaaaacaaggcgtttacgcgtggaaactaaagcttaatcaaaacggctttgtttataaagtagatgaaggccccgctccggatggcgaagagtactttgcgtttgcactacttaatgcctccgctcgttggggtaattcaggggagtttaactactacaacgatgcaattaccatgttaaacacaattaaaaataagctgatggaaaaccaaataatacgcttttcgccttacattgataacctaacagacccttcttaccatatacctgcgttttacgactactttgctaataacgtaactaaccaagcagacaaaacttactggcgacaagtagccacaaaaagtagaaccttactcaaaaaccattttacaaaagtaagtggtagcccgcattggaacttacctacatttttatcgcgcttagatggcagccctgttattggctacatttttaacggccaagcaaacccaggtcaatggtatgaatttgatgcatgccgcgtatttatgaatgtggggttagaggcgcattttatgggtgctcaagcgtggcataaaagtgcagttaataaagcactgggctttttaagttatgcaaaaacaaacaacagtaaaaactgttacgagcaagtgtattcgtacggtggagcgcaaaacagaggctgtgcaggcgaaggtcaaaaagccgcgaatgcagtagcgttacttgcttcaacaaatgctgggcaagcaaatgagttttttaacgaattttggtctttatcgcaaccaacgggtgactaccgttactataatggttcgttatatatgttagctatgctgcatgtatcgggcaattttaagttttataacaacacgtttaattaa

SEQ ID NO: 33 - protein, 405 aa, Artificial sequence
  Synthetic Construct
AFNNNPSSVGAYSSGTYRNLAQEMGKTNIQQKVNSTFDNMFGYNNTQQLYYPYTENGVYKAHYIKAINPDEGDDIRTEGQSWGMTAAIMLNKQEEFDNLWRFAKAYQKNPDNHPDVKKQGVYAWKLKLNQNGFVYKVDEGPAPDGEEYFAFALLNASARWGNSGEFNYYNDAITMLNTIKNKLMENQIIRFSPYIDNLTDPSYHIPAFYDYFANNVTNQADKTYWRQVATKSRTLLKNHFTKVSGSPHWNLPTFLSRLDGSPVIGYIFNGQANPGQWYEFDACRVFMNVGLEAHFMGAQAWHKSAVNKALGFLSYAKTNNSKNCYEQVYSYGGAQNRGCAGEGQKAANAVALLASTNAGQANEFFNEFWSLSQPTGDYRYYNGSLYMLAMLHVSGNFKFYNNTFN

SEQ ID NO: 34 - DNA, 1281 bp, Unknown
  Xylanase sequence obtained from microbial strain Sp.23.2bis
  CDS (1)..(1281)
atgaaagtattttttaaaataacaactttattgttaacgctaacaagctatcaatcacttgctgcatttaataataacccatcgagtgtaggcgccgacagttcaggagcataccgtaacctcgcacaagaaatgggtaaaacaaatatacagcaaaaagtgaatagtacttttgacaatatgtttggctataacaacacacaacaactttactacccgtacaccgaaaacggtgtttataaagcacactacataaaagcaattaacccagacgaaggcgacgatataagaacagaagggcaatcgtggggaatgactgccgctatcatgcttaataaacaagaagaatttgataatctatggcgctttgcaaaagcgtatcaaaaaaatccagacaaccaccctgatgtaaaaaaacaaggcgtttacgcgtggaaactaaagcttaatcaaaacggctttgtttataaagtagatgaaggccccgctccggatggcgaagagtactttgcgtttgcactacttaatgcctccgctcgttggggtaattcaggggagtttaactactacaacgatgcaattaccatgttaaacacaattaaaaataagctgatggaaaaccaaataatacgcttttcgccttacattgataacctaacagacccttcttaccatatacctgcgttttacgactactttgctaataacgtaactaaccaagcagacaaaacttactggcgacaagtagccacaaaaagtagaaccttactcaaaaaccattttacaaaagtaagtggtagcccgcattggaacttacctacatttttatcgcgcttagatggcagccctgttattggctacatttttaacggccaagcaaacccaggtcaatggtatgaatttgatgcatggcgcgtaattatgaatgtgggtttagaggcgcatttaatgggtgctcaagcatggcataaaagtgcagtaaataaagcattaggctttttaagttatgcaaaaacaaacaacagtaaaaactgttacgagcaagtgtattcgtacggtggagcgcaaaacaaaggttgtgcaggcgaaggtcaaaaagccgcgaatgcagtagcgttacttgcttcaacaaatgctgggcaagcaaatgaattttttaacgagttttggtctttatcgcaaccaacaggtaactaccgttactacaatggttcgctgtatatgttagctatgctgcatgtttcgggcaattttaagttttataacaacacgtttaattaa

SEQ ID NO: 35 - protein, 426 aa, Artificial sequence
  Synthetic Construct
MKVFFKITTLLLTLTSYQSLAAFNNNPSSVGADSSGAYRNLAQEMGKTNIQQKVNSTFDNMFGYNNTQQLYYPYTENGVYKAHYIKAINPDEGDDIRTEGQSWGMTAAIMLNKQEEFDNLWRFAKAYQKNPDNHPDVKKQGVYAWKLKLNQNGFVYKVDEGPAPDGEEYFAFALLNASARWGNSGEFNYYNDAITMLNTIKNKLMENQIIRFSPYIDNLTDPSYHIPAFYDYFANNVTNQADKTYWRQVATKSRTLLKNHFTKVSGSPHWNLPTFLSRLDGSPVIGYIFNGQANPGQWYEFDAWRVIMNVGLEAHLMGAQAWHKSAVNKALGFLSYAKTNNSKNCYEQVYSYGGAQNKGCAGEGQKAANAVALLASTNAGQANEFFNEFWSLSQPTGNYRYYNGSLYMLAMLHVSGNFKFYNNTFN

SEQ ID NO: 36 - DNA, 1218 bp, Unknown
  Xylanase sequence (mature protein) obtained from microbial strain Sp.23.2bis
  CDS (1)..(1218)
gcatttaataataacccatcgagtgtaggcgccgacagttcaggagcataccgtaacctcgcacaagaaatgggtaaaacaaatatacagcaaaaagtgaatagtacttttgacaatatgtttggctataacaacacacaacaactttactacccgtacaccgaaaacggtgtttataaagcacactacataaaagcaattaacccagacgaaggcgacgatataagaacagaagggcaatcgtggggaatgactgccgctatcatgcttaataaacaagaagaatttgataatctatggcgctttgcaaaagcgtatcaaaaaaatccagacaaccaccctgatgtaaaaaaacaaggcgtttacgcgtggaaactaaagcttaatcaaaacggctttgtttataaagtagatgaaggccccgctccggatggcgaagagtactttgcgtttgcactacttaatgcctccgctcgttggggtaattcaggggagtttaactactacaacgatgcaattaccatgttaaacacaattaaaaataagctgatggaaaaccaaataatacgcttttcgccttacattgataacctaacagacccttcttaccatatacctgcgttttacgactactttgctaataacgtaactaaccaagcagacaaaacttactggcgacaagtagccacaaaaagtagaaccttactcaaaaaccattttacaaaagtaagtggtagcccgcattggaacttacctacatttttatcgcgcttagatggcagccctgttattggctacatttttaacggccaagcaaacccaggtcaatggtatgaatttgatgcatggcgcgtaattatgaatgtgggtttagaggcgcatttaatgggtgctcaagcatggcataaaagtgcagtaaataaagcattaggctttttaagttatgcaaaaacaaacaacagtaaaaactgttacgagcaagtgtattcgtacggtggagcgcaaaacaaaggttgtgcaggcgaaggtcaaaaagccgcgaatgcagtagcgttacttgcttcaacaaatgctgggcaagcaaatgaattttttaacgagttttggtctttatcgcaaccaacaggtaactaccgttactacaatggttcgctgtatatgttagctatgctgcatgtttcgggcaattttaagttttataacaacacgtttaattaa

SEQ ID NO: 37 - protein, 405 aa, Artificial sequence
  Synthetic Construct
AFNNNPSSVGADSSGAYRNLAQEMGKTNIQQKVNSTFDNMFGYNNTQQLYYPYTENGVYKAHYIKAINPDEGDDIRTEGQSWGMTAAIMLNKQEEFDNLWRFAKAYQKNPDNHPDVKKQGVYAWKLKLNQNGFVYKVDEGPAPDGEEYFAFALLNASARWGNSGEFNYYNDAITMLNTIKNKLMENQIIRFSPYIDNLTDPSYHIPAFYDYFANNVTNQADKTYWRQVATKSRTLLKNHFTKVSGSPHWNLPTFLSRLDGSPVIGYIFNGQANPGQWYEFDAWRVIMNVGLEAHLMGAQAWHKSAVNKALGFLSYAKTNNSKNCYEQVYSYGGAQNKGCAGEGQKAANAVALLASTNAGQANEFFNEFWSLSQPTGNYRYYNGSLYMLAMLHVSGNFKFYNNTFN